(19) United States
(12) Patent Application Publication
(10) Pub. No.: US 2023/0144704 A1
(43) Pub. Date: May 11, 2023

(54) TAILORED HYPOIMMUNE NANOVESICULAR DELIVERY SYSTEMS FOR CANCER TUMORS, HEREDITARY AND INFECTIOUS DISEASES

(71) Applicants: Thomas MALCOLM, Andover, NJ (US); Safia RIZVI, Jamaica Plain, MA (US); Surya SANKURATRI, Newark, CA (US)

(72) Inventors: Thomas MALCOLM, Andover, NJ (US); Safia RIZVI, Jamaica Plain, MA (US); Surya SANKURATRI, Newark, CA (US)

(21) Appl. No.: 17/978,082

(22) Filed: Oct. 31, 2022

Related U.S. Application Data
(60) Provisional application No. 63/297,115, filed on Jan. 6, 2022.
(60) Provisional application No. 63/276,325, filed on Nov. 5, 2021.

(51) Int. Cl. A61K 47/69; C12N 5/074; A61K 9/50; C07K 14/725

(52) U.S. Cl. CPC A61K 47/6901; C12N 5/0696; A61K 9/5068; C07K 14/7051; C12N 2506/45; C12N 2510/00; C07K 2319/33; C07K 2319/03

(57) ABSTRACT

Hypoimmunogenic induced pluripotent stem cell (iPSC)-derived biomimetic nanovesicles (Hypo-BioNVs) or Hypo-exosomes including tailored chimeric antigen receptor (CARs) which can recognize target biomarkers. A method of making Hypo-BioNVs. A method of treating an individual with cancer, by administering the Hypo-BioNVs to an individual, targeting cancer cells, and treating the cancer. Hypo-BioNVs including tailored CARs which can recognize target biomarkers through a VERR including viral receptors of an oncolytic virus. A method of treating an individual with cancer, by administering Hypo-BioNVs including CAR receptors to an individual, targeting cancer cells, and treating the cancer. A method of targeting cells in an individual, by administering the Hypo-BioNVs to an individual, and targeting cells to be destroyed or treated for cancer tumors (both liquid and solid), infectious disease, hereditary conditions, autoimmune disease, or metabolic disorders.

### BACKGROUND OF THE INVENTION

### 1. Technical Field

The present invention relates to compositions and methods for delivering therapeutics and gene editing compounds.

### 2. Background Art

Effectively targeting in vivo delivery of gene edited therapeutics or gene editors to diseased cells remains one of the greatest challenges in modern biotechnology. Even with recent improvements, delivery mechanisms consistently have inhibitory issues such as inconsistent and low frequency targeting, low targeting interchangeability/capacity, lack of non-autologous approaches, high immune neutralization, high manufacturing costs, regulatory roadblocks (FDA clinical holds), low ability to scale/collection/expansion, packaging size constraints, inefficient packaging, organ sinking (liver and marrow), and low half-life of delivery vehicle.

AAV (adeno-associated virus) is commonly used as a vector for gene editing therapeutics. AAV has a size of approximately 22 nm. AAV has high liver sinking properties (though lower with new mutants), dosing limitations due to pre- and post-dosing anti-drug antibodies (though higher with new serotypes), cargo-insert size limitations of approximately 1100 amino acids, limited serotype scalability and targeting efficiency is generally limited by serotype tropism.

Liposomes can also be used as a delivery mechanism. Liposomes are 50-1000 nm in size. Organ sinking and neutralizing effects with liposomes include RES (reticuloendothelial system), EPR (enhanced permeability and retention), ABC (accelerated blood clearance), CARPA (complement activation-related pseudoallergy), and opsonization. Immune neutralization varies and is ligand dependent. There are no size constraints, but expensive active loading is necessary with most compounds. Manufacturing can also involve ligand addition or PEGylation. Liposomes are ligand targeted and have poor solid tumor penetration.

Tunable dendrimers are 1.5 to 10 nm. They are highly interactive with blood proteins and have increased IgG macrophage Fc clearance. Packaging constraints include being externally conjugated nucleic acids. Tunable dendrimers have not been tested widely for use with large biologic therapies, including gene editors.

Polymeric micelles are 10-100 nm in size. There are no known organ sinking or neutralization effects or immune neutralization. There may not be size constraints and they have not been tested widely for gene editing. They are ligand targeted and stimuli inducible/releasable.

Exosomes are 30-150 nm in size. Their organ sinking and neutralization effects are high but can be lower with new targeted mutants. They have low immunogenicity, but the degree varies with each ligand. There are no size constraints, but they are very inefficient with loading. It is also difficult to produce therapeutic quantities as most manufacturing methods rely on cellular shedding mechanisms. Generally, they are ligand targeted with average tumor penetration.

CAR T-cells have also been used to target cells to treat cancer. CAR T-cell therapy is a cancer therapy that requires the collection of a patient's own immune cells (T cells) to treat their cancer. T-cells normally attack invasive microorganisms, but in CAR T-cell therapy, the T-cells are reengineered to attack cancer cells. First, T-cells are separated from the patient's blood and genetically engineered to produce chimeric antigen receptors (CARs) on their surface that allow the T-cells to attach to a specific tumor antigen. The CARs do not exist naturally and are made up of fragments of synthetic antibodies. CARs rely on engineered signaling and co-stimulatory domains inside the T-cell to function.

Once the CAR T-cells have been produced, they are expanded to produce large quantities that can then be infused back into the patient. Generally, the patient has undergone chemotherapy to deplete their lymphocytes prior to the infusion. The CAR T-cells are attracted to the tumor antigens on the cancer cells they are designed for and kill the cancer cells that have those specific antigens.

CAR T-cell therapies have been approved for the treatment of acute lymphoblastic leukemia (ALL) in children and advanced lymphomas in adults. For example, CAR T-cells that target CD-19 (tisangenlecleucel, KYMRIAH™, Novartis) have been approved to treat ALL. YESCARTA™ (axicabtagene ciloleucel, Gilead/Kite Pharmaceuticals) is approved for the treatment of lymphomas. Studies have also been conducted to target CD-22 in cells that have lost CD-19 expression. Dual targeting of CD-19 and CD-123 in leukemia has also been studied. CAR T-cells that target B cell maturation antigen (BCMA) have recently been approved as the treatment of multiple myeloma (MM). It is unclear currently whether CAR T-cells can treat solid tumors due to the microenvironment that surrounds them, but studies are being performed with targeting mesothelin expressed on pancreatic and lung cancers, EGFRvIII expressed on glioblastoma, and other tumor specific markers expression on solid tumors.

There are several drawbacks to CAR T-cell therapy. It can cause cytokine release syndrome (CRS) that results in high fevers and low blood pressure. This can require additional treatment with blocking IL-6 activity. It can also cause B cell die off (B cell aplasia) and require further treatment with immunoglobulins to provide antibodies. Other side effects include cerebral edema and neurotoxicity. Patients may also not have enough T-cells to harvest and engineer. Furthermore, multiple rounds of treatment are often required, especially when tumor cells lose antigen expression.

Another delivery vehicle is biomimetic exosomes or nanovesicles (BioNVs) or biofunctionalized liposome-like nanovesicles (BLNs), which are biologically-derived nano-sized vesicles that are similar to exosomes but larger. BioNVs have been successfully used for treatment of cancer cells with passive targeting (accumulation in tumoral tissue) (35) active targeting (functionalized nanovesicles recognize receptors in tumor cells) most recently—Zhang et al 2018 (39), Goh et al 2017 (17, 18), and Lunavat et al 2016 (22). Further, they have been successfully used for testing anti-inflammatory properties (α4β7 integrin—implications inflammatory bowel disease (4)), and hepatocyte proliferation when derived from primary hepatocytes (35). Molinaro et al 2018 (25) incorporated membrane proteins within the bilayer of biomimetic nanovesicles using a microfluidic-based platform, which extended shelf-life and retained biological functions of donor cells. Jang et al 2013 (19) developed bioinspired exosome-mimetic nanovesicles that deliver chemotherapeutics to the tumor tissue after systemic administration, produced by the breakdown of monocytes or macrophages using a serial extrusion through filters with diminishing pore sizes. The nanovesicles have shown natural targeting ability of cells by maintaining the topology of plasma membrane proteins, as well as targeted delivery of chemotherapeutics to tumor tissue and reduced tumor growth.

There are several advantages to using BioNVs over exosomes. BioNVs are much easier to manufacture than collecting exosomes (which are limited when harvested from patients). BioNVs are therefore far more scalable and much easier to manufacture.

BioNVs are an option of a delivery vehicle for CRISPR Cas9 systems (U.S. Patent Application Publication Nos. 20160281111, 20170022507, 20180119123, 20180155789, 20180236103, and 20180251770). BioNVs can also be engineered by CRISPR (U.S. Patent Application Publication No. 20190085284). U.S. Patent Application Publication No. 20190060483 to Dooley et al discloses methods of purification of nanovesicles, and the purification can be related to surface proteins on nanovesicles and/or exosomes.

There remains a need for an allogenic, highly targeted, tumor microenvironment-accessible therapeutic delivery system with low-probability of CRS, and that can harness the power of activated whole cells and address the challenges associated with whole cell CAR-based (or similar, such as a T-Cell Receptor-based (TCR) or TRUCK engineered or engineered CAR-NK cell) therapies to treat cancer tumors (both liquid and solid), infectious disease, hereditary conditions, autoimmune disease, or metabolic disorders.

### SUMMARY OF THE INVENTION

The present invention provides for a novel method of generating allogenic biomimetic nanovesicles (BioNVs) from gene edited iPSCs by disrupting cell membranes of the gene edited iPSCs using a method of sonicating, adaptive focused acoustics technology, extrusion, serial extrusion, rupturing by detergents or enzymes, electroporation, or any combination of these methods, then purifying the BioNVs by a method of microfiltration, affinity chromatography, size exclusion chromatography, gel purification, centrifugation, or combinations thereof.

The present invention provides for BioNVs produced by the above method(s).

The present invention provides for a composition of a therapeutic agent packaged in the BioNVs for treatment of disease.

The present invention provides for a method of generating BioNVs with targeting capabilities by disrupting cell membranes of genetically engineered iPSCs that contain a targeting surface marker by a method of sonicating, adaptive focused acoustics technology, extrusion, serial extrusion, rupturing by detergents or enzymes, or electroporation.

The present invention provides for BioNVs with targeting capabilities.

The present invention also provides for a composition of a therapeutic agent packaged in BioNVs with targeting capabilities.

The present invention provides for hypoimmunogenic induced pluripotent stem cell (iPSC)-derived biomimetic nanovesicles (Hypo-BioNVs) including tailored chimeric antigen receptor (CARs) which can recognize target cancer biomarkers through: 1) an antibody fragment scFV region, or bifunctional or ByTE antibodies 2) or by a viral epitope recognition receptor (VERR) derived from oncolytic viral receptors, 3) or by a camelid-derived variable heavy chain IgG fragment called a V_{H}H single-domain nanobody (V_{H}H nanobody), 4) or by a cartilaginous fish-derived variable heavy chain IgG fragment called a Variable New Antigen Receptor (V_{NAR}), 5) or by an engineered TCR, 6) or by any single heavy chain IgG fragment from which a variable region can be engineered into a CAR structure to recognize any biomarker, 7) or by affilins (or other affinity targeted [bio]chemical ligands), 8) or by chimeric endocrine receptors (CERs). An example of a VERR can be the vp1, vp2, or vp3 of SVV that targets TEM8 on various cancer cells. An example of a V_{H}H nanobody or V_{NAR} can be a single heavy chain domain that recognizes CD19, PD-L1, or EIIIB. All the above can be used in CAR design, and in multiple combinations (including a bispecific recognition method) to allow Hypo-BioNV targeting of any specific cell of interest. The Hypo-BioNV can also encapsulate and deliver any biologic drug or small molecule drug of choice.

The present invention provides for a method of making Hypo-BioNVs.

The present invention provides for a method of treating an individual with cancer, an infectious disease, hereditary disease, autoimmune disease, or metabolic disorders by administering the Hypo-BioNVs to an individual, targeting: 1) cancer cells, 2) cells that have been biochemically or genetically corrupted by (but not limited to) an infectious pathogen such as a virus, bacteria, or fungus, 3) cells that have hereditary aberrations or genetic mutations, and treating the cancer, infectious disease, hereditary disease, autoimmune disease, or metabolic disorders.

The present invention provides for Hypo-BioNVs including tailored CARs which can recognize target biomarkers through an scFv, VERR (that may include viral receptors from oncolytic viruses), a V_{H}H nanobody, a V_{NAR}, an affilin (or other affinity targeted [bio]chemical ligands), or a CER. A TCR can also be used in place of (or in combination with) a CAR.

The present invention provides for a method of treating an individual with cancer, an infectious disease, or hereditary disease, by administering Hypo-BioNVs including CAR (or TCR) receptors having an scFv, VERR (that may include viral receptors from oncolytic viruses), a V_{H}H nanobody, a V_{NAR}, an affilin (or other affinity targeted [bio]chemical ligands), or a CER to target individual's specific cancer cells, cells that have been infected by a pathogen or genetically defective cells, and treating the cancer, infectious disease, hereditary disease, autoimmune disease, or metabolic disorders.

The present invention provides for a method of targeting cells in an individual, by administering the Hypo-BioNVs to an individual, and targeting cells that need to be destroyed or treated.

The present invention provides for a method of treating an individual with cancer, by administering Hypo-BioNVs including CAR receptors having an scFv light and heavy chain of an antibody connected through peptide linker that can be adjusted/modified to any length to optimize the targeting efficiency, precision, specificity, selectivity, and robustness of the receptor's epitope to the biomarker target, to an individual, targeting cancer cells, and treating the cancer (and/or hereditary diseases, rare diseases, infectious diseases, autoimmune disease, or metabolic disorders).

The present invention provides a method for the ‘tunable’ expression of CARs on the surface of the iPSC(s) or any cell that is differentiated from the iPSC(s), so that the density of the CARs on the surface of the Hypo-BioNV that is derived from the iPSC(s) or any cell type that is differentiated from the iPSC(s) can be regulated. The resulting engineered Hypo-BioNV can be used in an individual, targeting their specific cancer cells, and treating the cancer (and/or hereditary diseases, rare diseases, infectious diseases, autoimmune disease, or metabolic disorders).

The present invention utilizes an integrated CRISPRa/3× g RNA expression system that is regulated by a Tetracycline on/off promoter (or any similar type of drug regulated promoter). Once the CRISPRa/3× gRNA system is expressed (through the addition/subtraction of tetracycline) the three gRNA(s) and CRISPRa selectively bind to the promoters of the upstream transcriptional activators for antibody expression (an event that occurs in B-cells). The transcriptional activators are Drm2, Bxp2, and Fr5. Once these three transcriptional activators are expressed, they bind to and activate the expression of an antibody gene within any given locus.

The present invention replaces the antibody gene at any given locus by a CAR cassette that has CRISPR gene editing gRNA sites engineered into the variable region of the CAR (or TCR) structure, allowing for rapid exchange of any type of variable epitope to target any biomarker. These variable regions can be an scFv, VERR, V_{H}H nanobody, V_{NAR}, an affilin (or other affinity targeted [bio]chemical ligands), or a CER, or any heavy chain single variable region. The resulting engineered Hypo-BioNV can be used in an individual, targeting specific cancer cells, and treating the cancer (and/or hereditary diseases, rare diseases, infectious diseases, autoimmune disease, or metabolic disorders).

The present invention allows for the tetracycline (or equivalent) regulation of CAR-density on the surface of the iPSC(s) or any cell type that is differentiated from the iPSC(s), so that the CAR density on resulting Hypo-BioNVs can be regulated or ‘tuned’. The resulting engineered Hypo-BioNV can be used in an individual, targeting their specific cancer cells, and treating the cancer (and/or address any other cellular defect arising from hereditary diseases, rare diseases, infectious diseases, autoimmune disease, or metabolic disorders).

The present invention includes a strategy for using these Tunable mini-CAR iPSC-derived Hypo-BioNVs (that may be loaded with a biologic or small molecule therapeutic) to target cancer cells in the stroma, neoplastic endothelial cells of the neovasculature that surrounds cancer solid tumors, as well as the cancer cells within the tumor themselves.

The present invention includes a strategy for using these Tunable mini-CAR iPSC-derived Hypo-BioNVs (that may be loaded with a biologic or small molecule therapeutic) as a co-therapeutic with whole cell CAR therapeutics (including but not limited to T-cells, Natural Killer cells, or macrophage).

The present invention includes a strategy for the Tunable mini-CAR iPSC-derived Hypo-BioNVsv (that may be loaded with a biologic or small molecule therapeutics, their precursor and/or genetically engineered to express molecules to defend against tumor micro-environment such as anti-checkpoint inhibition and metabolic stimulators such as cytokines) to attack solid tumors, while the whole cell CAR therapeutics (T-cells, Natural Killer cells, or macrophages for example, but not limited to these cells) destroy or treat any cells that are shed from the solid tumor (Circulating Tumor Cells—CTCs).

The present invention captures all biomarkers for the integration into the Tunable mini-CAR iPSC-derived Hypo-BioNVs (that may be loaded with a biologic or small molecule therapeutic), for the treatment of cancers, hereditary diseases, rare diseases, infectious diseases, autoimmune disease, or metabolic disorders with high degree of target specificity.

The present invention involves the antigen-mediated activation of lymphocyte cells (T-cells, Natural Killer, and macrophage, but not limited to these cells) that have been derived from iPSC(s) that express the desired CAR, using the methods for CAR expression in the engineered iPSC cell lines described above. After the base iPSC(s) (that contain all the engineering described above) has been differentiated into the desired lymphocyte cell line, the lymphocyte cell line is activated using the antigen that recognizes the engineered/targeted CAR. The antigen can be added in scaling concentrations between 0%, 10%, 20%, 30%, 40%, 50%, 60%, 70%, 80%, 90, 100% to achieve the best activation-to-CAR blocking ratio.

In another embodiment the activation of lymphocyte cells (T-cells, Natural Killer, and macrophage, but not limited to these cells) may occur with an ‘activating virus’ or ‘activating viral protein’ through an alternate receptor other than the engineered/targeted CAR (for example a naturally occurring TCR or viral receptor).

In another embodiment the activation of lymphocyte cells (T-cells, Natural Killer, and macrophage, but not limited to these cells) can occur using a combination of the antigen that recognizes the CAR and a viral antigen (as those described further below).

The present invention entails the disruption of the specific targeted antigen-activated (activated through the engineered CAR receptor, or an alternate receptor such as TCR, or other viral receptors, by using a ‘viral activator’) lymphocytes (T-cells, Natural Killer cells, macrophage, but not limited to these cells) to create Hypo-BioNVs that encapsulate the cytokines, chemokines and cytotoxic biomolecules that normally accompany whole activated lymphocyte cells. The resulting Hypo-BioNVs therefore mimic activated whole cell lymphocytes, but do not contain genetic information that could lead to issues such as cytokine storms or teratoma formations.

The present invention entails the regulation of the concentrations of Interleukins within the T-cell that are related to T-cell recruitment, the prevention of T-cell exhaustion at the site of the solid tumor, T-cell effector function and recruitment and the prevention of CRS. The regulation of the interleukins occurs in the whole T-cell prior to Hypo-BioNV derivation, thereby ensuring the correct concentration of these Interleukins by and within the Hypo-BioNVs to the site of the solid tumor.

### DESCRIPTION OF THE DRAWINGS

Other advantages of the present invention are readily appreciated as the same becomes better understood by reference to the following detailed description when considered in connection with the accompanying drawings wherein:

FIG. 1 is a schematic of derivation of biomimetic nanovesicles of the present invention;

FIG. 2 is an example of a hypoimmunogenic iPSC-derived biomimetic nanovesicle (Hypo-BioNV) for use in cancer and infectious disease treatment;

FIG. 3 is an example of a Hypo-BioNV for use in gene editing;

FIG. 4 is a schematic showing the use of CRISPRa regulated with a Tetracycline on/off promoter to activate upstream transcriptional activators that when expressed activate a downstream integrated CAR cassette (containing CRISPR directed swap sites for any scFv, VERR, or V_{H}H nanobody) strategically placed in an antibody locus (antibody locus swapped out).;

FIG. 5 is a schematic of two different CAR structures (primary and secondary generation) that can be used to coat the Hypo-BioNVs;

FIG. 6 is a schematic of two paths of Hypo-BioNVs to treat cancer and infectious diseases;

FIG. 7 is a schematic of two paths of Hypo-BioNVs to encapsulate either post-loaded DNA plasmids, or pre-loaded (via expression cassettes) gene editing molecules (nucleases/gRNAs) for treating diseases with a gene editing approach;

FIG. 8 is a schematic of a manufacturing process;

FIG. 9 is a graph of an example of size distribution via purification;

FIG. 10 shows examples of Hypo-BioNVs that may be engineered, manufactured and purified (but not limited to these examples);

FIG. 11A is a representation of a 2^{nd} generation CAR receptor, FIG. 11B is a representation of a 2^{nd} generation CAR with various combinations of VERRs, and FIG. 11C is a representation of a CAR with various combinations of scFvs and VERRs;

FIG. 12 is a representation of a 2^{nd} generation CAR receptor that replaces the scFv or VERR (and linker) with a variable heavy chain IgG fragment called a V_{H}H nanobody or an Immunoglobulin New Antigen Receptor (IgNAR) variable region called a V_{NAR};

FIG. 13 is a diagram outlining two strategies for using tunable mini-CAR Hypo-BioNVs to treat solid tumor cancers;

FIG. 14 is a diagram of strategy for fusing Hypo-BioNV lipid bilayer with plasma membrane of a targeted cell using an acidic activated fusion protein model;

FIG. 15 is a diagram of strategy for fusing Hypo-BioNV lipid bilayer with plasma membrane of a targeted cell using a viral receptor (such as gp120/gp41 of HIV) membrane fusion protein model;

FIG. 16 is a diagram of strategy for fusing Hypo-BioNV lipid bilayer with plasma membrane of a targeted cell using a CAR-target-activated fusion protein model;

FIG. 17 is a diagram of strategy for using HIV gp120/gp41 receptor ligand complexes expressed on the surface of a Hypo-BioNV for the targeted fusion to the cell of interest, where the deliverable payload is directly injected into the cytoplasm of the cell, avoiding the endosomal pathway;

FIG. 18 is a diagram of strategy for creating a standard curve for immunological synapse quality; and

FIG. 19 is a diagram of strategy for testing BioNVs in an animal model for efficacy.

### DETAILED DESCRIPTION OF THE INVENTION

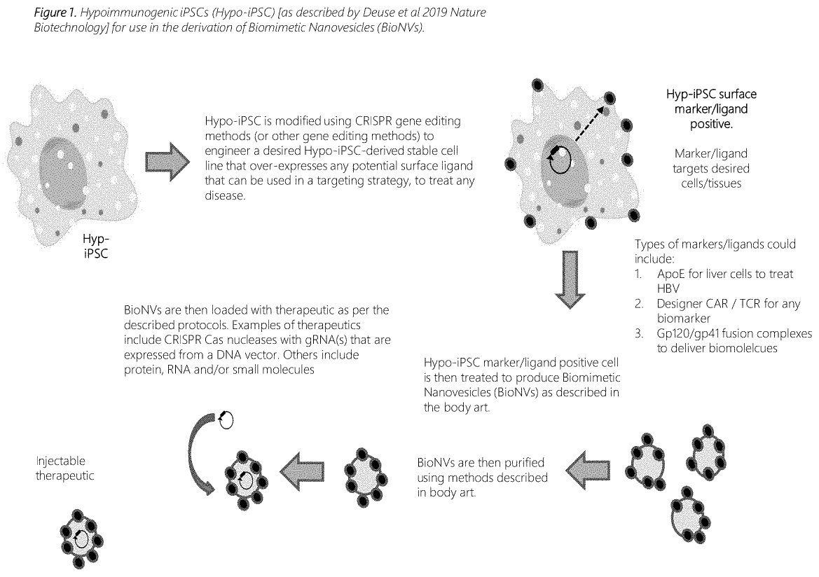

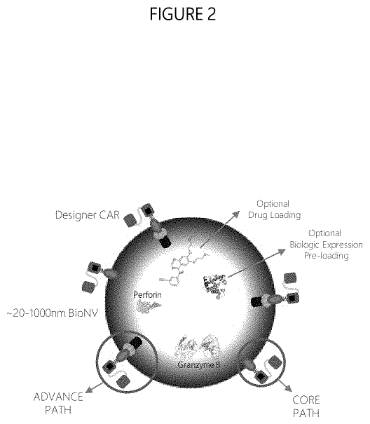

The present invention provides for hypoimmunogenic induced pluripotent stem cell (iPSC)-derived biomimetic nanovesicles (Hypo-BioNVs) with tailored chimeric antigen receptor (CARs) (or T-Cell Receptor TCR)) on the surface to recognize one target or multiple biomarkers through an antibody fragment scFV region for a desired/specific cancer biomarker with bifunctional or ByTE antibodies or by a viral epitope recognition receptor (VERR) or by a variable heavy chain IgG fragment V_{H}H or V_{NAR} or through a T-Cell Receptor (TCR) or by a chimeric endocrine receptor (CER). Additionally, the density of the CARs on the surface of the iPSC(s) or any cell that is differentiated from the iSPC(s), and the resulting Hypo-BioNVs that are derived from the iPSC(s) can be regulated using a tetracycline on/off promoter (or similar drug regulated promoters) to drive the expression of a CRISPR activation/gRNA (CRISPRa) system. The CRISPRa system then activates the antibody-regulating transcription factors Drm2, Fr5, and Bxp2, that regulate the expression of an engineered CAR-cassette that has been integrated at the site of an antibody locus (where the antibody genes have been replaced). All variations of an scFv, VERR, V_{H}H nanobodies, V_{NARs}, affilins (or other affinity targeted [bio]chemical ligands), and CERs can be used in CAR or TCR design to allow Hypo-BioNV targeting of any cell of interest (targeting any type of biomarker). The Hypo-BioNV can also encapsulate and deliver any small molecule, biologic, nucleic, and/or gene editing therapeutic of choice to any intended cellular targets and treat diseases, especially those caused by hereditary aberrations/mutations or pathogens such as viruses, bacteria, and fungus. A method of making a Hypo-BioNV is shown in FIG. 1 and an example of the Hypo-BioNVs is shown in FIG. 2.

“BioNV and Hypo-BioNV” as used herein, refers to biologically-derived nano-sized vesicles that can have designed biological functionalization.

“iPSC” as used herein refers to induced pluripotent stem cells, which are stem cells that can be generated directly from adult cells. iPSCs can propagate indefinitely and can become any cell type in the body.

“CER” as used herein refers to a chimeric endocrine receptor. It replaces the antigen receptor (scFV region of CAR) with an endocrine recognition domain instead. The predominant biomarker target is Follicle Stimulating Hormone (FSH) receptor. FSH is predominantly expressed on ovarian cancer cells and CERs can be used to target cancer cells. WO2016054153 discloses a nucleic acid sequence that encodes a chimeric protein comprising a ligand that comprises a naturally occurring or modified follicle stimulating hormone sequence, e.g., an FSHp sequence, or fragment thereof, which ligand binds to human follicle stimulating hormone (FSH) receptor, linked to either (a) a nucleic acid sequence that encodes an extracellular hinge domain, a transmembrane domain, a co-stimulatory signaling region, and a signaling endodomain; or (b) a nucleic acid sequence that encodes a ligand that binds to NKG2D. The vector containing the nucleic acid sequence, the chimeric proteins so encoded, and modified T cells expressing the chimeric protein, as well as method of using these compositions for the treatment of FSHR-expressing cancers or tumor cells are also provided.

“Affilin” as used herein refers to a protein derived from ubiquitin or gamma-B crystallin that selectively binds antigens. They are essentially antibody mimetics.

The term “vector” includes cloning and expression vectors, as well as viral vectors and integrating (or non-integrating) vectors. An “expression vector” is a vector that includes a regulatory region. Vectors are also further described below.

The term “lentiviral vector” includes both integrating and non-integrating lentiviral vectors.

Viruses replicate by one of two cycles, either the lytic cycle or the lysogenic cycle. In the lytic cycle, first the virus penetrates a host cell and releases its own nucleic acid. Next, the host cell's metabolic machinery is used to replicate the viral nucleic acid and accumulate the virus within the host cell. Once enough virions are produced within the host cell, the host cell bursts (lysis), and the virions go on to infect additional cells. Lytic viruses can integrate viral DNA into the host genome as well as be non-integrated where lysis does not occur over the period of the infection of the cell.

“Lysogenic virus” as used herein, refers to a virus that replicates by the lysogenic cycle (i.e., does not cause the host cell to burst and integrates viral nucleic acid into the host cell DNA). The lysogenic virus can mainly replicate by the lysogenic cycle but sometimes replicate by the lytic cycle. In the lysogenic cycle, virion DNA is integrated into the host cell, and when the host cell reproduces, the virion DNA is copied into the resulting cells from cell division. In the lysogenic cycle, the host cell does not burst. Lysogenic viruses treated with the compositions and methods of the present invention can include, but are not limited to, hepatitis A, hepatitis B, hepatitis D, HSV-1, HSV-2, cytomegalovirus, Epstein-Barr virus, Varicella Zoster virus, HIV1, HIV2, HTLV1, HTLV2, Rous Sarcoma virus, HPV virus, yellow fever, zika, dengue, West Nile, Japanese encephalitis, lyssa virus, vesiculovirus, cytohabdovirus, Hantaan virus, Rift Valley virus, Bunyamwera virus, Lassa virus, Junin virus, Machupo virus, Sabia virus, Tacaribe virus, Flexal virus, Whitewater Arroyo virus, ebola, Marburg virus, JC virus, and BK virus.

“Lytic virus” as used herein refers to a virus that replicates by the lytic cycle (i.e., causes the host cell to burst after an accumulation of virus within the cell). The lytic virus can mainly replicate by the lytic cycle but sometimes replicate by the lysogenic cycle. Lytic viruses treated by the compositions and methods of the present invention can include, but are not limited to, hepatitis A, hepatitis C, hepatitis D, coxsachievirus, HSV-1, HSV-2, cytomegalovirus, Epstein-Barr virus, varicella zoster virus, HIV1, HIV2, HTLV1, HTLV2, Rous Sarcoma virus, rota, seadornvirus, coltivirus, JC virus, and BK virus.

The compositions of the present invention can be used to treat infections caused by either active or latent viruses. The compositions of the present invention can be used to treat individuals in which latent virus is present, but the individual has not yet presented symptoms of the virus. The compositions can target virus in any cells in the individual, such as, but not limited to, CD4+ lymphocytes, macrophages, fibroblasts, monocytes, T lymphocytes, B lymphocytes, natural killer cells, dendritic cells such as Langerhans cells and follicular dendritic cells, hematopoietic stem cells, endothelial cells, brain microglial cells, and gastrointestinal epithelial cells.

“gRNA” as used herein refers to guide RNA. The gRNAs in the CRISPR Cas systems and other CRISPR nucleases herein are used for engineering CAR T cells. This is accomplished by using one or more specifically designed gRNAs to avoid the issues seen with single gRNAs such as mutations. The gRNA can be a sequence complimentary to a coding or a non-coding sequence and can be tailored to the particular sequence to be targeted. The gRNA can be a sequence complimentary to a protein coding sequence, for example, a sequence encoding one or more viral structural proteins. The gRNA sequence can be a sense or anti-sense sequence. It should be understood that when a gene editor composition is administered herein, preferably (but not limited to) this includes two or more gRNAs; however, a single gRNA can also be used.

“Nucleic acid” as used herein, refers to both RNA and DNA, including cDNA, genomic DNA, synthetic DNA, and DNA (or RNA) containing nucleic acid analogs, any of which may encode a polypeptide of the invention and all of which are encompassed by the invention. Polynucleotides can have essentially any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA) and portions thereof, transfer RNA, ribosomal RNA, siRNA, micro-RNA, short hairpin RNA (shRNA), interfering RNA (RNAi), ribozymes, cDNA, recombinant polynucleotides, branched polynucleotides, plasmids, vectors, isolated DNA of any sequence, isolated RNA of any sequence, nucleic acid probes, and primers, as well as nucleic acid analogs. In the context of the present invention, nucleic acids can encode a benign surface marker whose expression is regulated by viral (for clearing virally infected cells) or epigenetic regulatory elements (for clearing cancer cells)

An “isolated” nucleic acid can be, for example, a naturally occurring DNA molecule or a fragment thereof, provided that at least one of the nucleic acid sequences normally found immediately flanking that DNA molecule in a naturally occurring genome is removed or absent. Thus, an isolated nucleic acid includes, without limitation, a DNA molecule that exists as a separate molecule, independent of other sequences (e.g., a chemically synthesized nucleic acid, or a cDNA or genomic DNA fragment produced by the polymerase chain reaction (PCR) or restriction endonuclease treatment). An isolated nucleic acid also refers to a DNA molecule that is incorporated into a vector, an autonomously replicating plasmid, a virus, or into the genomic DNA of a prokaryote or eukaryote. In addition, an isolated nucleic acid can include an engineered nucleic acid such as a DNA molecule that is part of a hybrid or fusion nucleic acid. A nucleic acid existing among many (e.g., dozens, or hundreds to millions) of other nucleic acids within, for example, cDNA libraries or genomic libraries, or gel slices containing a genomic DNA restriction digest, is not an isolated nucleic acid.

Isolated nucleic acid molecules can be produced by standard techniques. For example, polymerase chain reaction (PCR) techniques can be used to obtain an isolated nucleic acid containing a nucleotide sequence described herein, including nucleotide sequences encoding a polypeptide described herein. PCR can be used to amplify specific sequences from DNA as well as RNA, including sequences from total genomic DNA or total cellular RNA. Various PCR methods are described in, for example, PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >50-100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids of the invention also can be obtained by mutagenesis of, e.g., a naturally occurring portion of a Cas9-encoding DNA (in accordance with, for example, the formula above).

More specifically, the present invention provides for a method of generating bionanovesicles (BioNVs) from gene-edited iPSCs by disrupting cell membranes of the gene edited iPSCs via sonicating, adaptive focused acoustics technology, extrusion, serial extrusion, rupturing the cells by detergent or by enzymes (such as by trypsinization), or using electroporation. It should be understood that while sonication is further referred to below, any of the above methods can be used in disrupting cell membranes. BioNVs are generally membranes enclosing an internal space that can be used for transporting therapeutic agents. The method includes inducing vesicle budding with mild detergent treatment in a shaker, low-speed centrifugation to collect the vesicles, and Covaris sonication (vesicle sizing and loading). Analysis can be performed with Malvern Zetasizing and flow cytometry. The present invention also provides for the BioNVs produced by this method. The BioNVs can be 20-1000 nm in diameter, but not limited to this size range.

Most preferably, the gene-edited iPSCs are CRISPR modified iPSCs and hypoimmunogenic (Hyp-iPSCs), such as those described in Deuse et al 2019 (8). These iPSCs have been modified to inactivate MHC class I and II genes and over-express CD47 such that the resulting iPSCs are allogenic and do not cause an immune reaction in patients they are administered to. Therefore, BioNVs derived from such iPSCs are useable in all patients. Various gene editing methods (further described below) can be used to create the iPSC cells instead of CRISPR/Cas9, such as, but not limited to, TALENs, ZFNs, RNase P RNA, C2c1, C2c2, C2c3, Cas9, Cpf1, TevCas9, Archaea Cas9, CasY.1, CasY.2, CasY.3, CasY.4, CasY.5, CasY.6, CasX, Cas Omega, or Saliogase. As shown in FIG. 1, Hypo-iPSCs are modified using CRISPR or other gene editing methods to engineer a desired Hypo-iPSC-derived stable cell line that overexpresses any potential surface ligand that can be used in a targeting strategy to treat any disease. The Hypo-iPSCs can include surface markers or ligands that target desired cells or tissues, such as, but not limited to, HIV gp120/gp41 to target CD4+ cells, ApoE for liver cells to treat HBV, TCRs or CARs (with interchangeable regions called scFv, VERRs, V_{H}H, V_{NAR}, affilins (or other affinity targeted [bio]chemical ligands), or CERs) for various cancer, hereditary disease and/or pathogen-infected cellular targets, autoimmune disease, or metabolic disorders.

The density of such surface ligands can be regulated or ‘tuned’ chemically, as described above. The Hypo-iPSC marker/ligand positive cells are then sonicated (or serially extruded) to shear the cells to produce Hypo-BioNVs, then purified by a method of either microfiltration, affinity chromatography, size exclusion chromatography, gel purification, centrifugation, or combinations thereof, and 1) loaded with therapeutics (such as, but not limited to, CRISPR Cas nuclease with gRNA(s) that are expressed from a DNA vector, protein, RNA, and/or small molecules); and 2) Pre-loaded by expressing CRISPR Cas nucleases and/or gRNAs from a chromosomally-integrated and stable ‘gene editing cassette’ (that can be regulated by drugs such as tetracycline—Tet On/Off systems or equivalents). The result is an injectable therapeutic.

The Hypo-BioNVs are therefore scalable using the sonication (or serial extrusion) process and no longer personalized (autologous). Previous methods deriving BioNVs from PBMCs (using the sonication AFA method) from individual patients provided a personalized approach of BioNV manufacture and development, and this method would be limited to individual patients due to immune-responses that would occur in cross patient populations, and therefore commercially not viable. The present invention solves this problem by providing Hypo-BioNVs that can be used in all patient populations.

The present invention has several advantages over the prior art. By using allogeneic iPSCs as the source for BioNV derivation, the resulting Hypo-BioNVs can be engineered to be loaded with any ligand that can target a desired receptor. The allogeneic iPSC can be engineered to over-express any ligand that in turn can recognize any receptor. Once the ligand-loaded allogeneic iPSC is engineered, the Hypo-BioNVs are then derived from the cell line, packaged with the correct vector therapeutic (such as CRISPR Cas nucleases and gRNAs on a DNA vector), and delivered to the targeted cell.

The present invention also provides for a composition of a therapeutic agent packaged in Hypo-BioNVs for treatment of disease, such as diseases caused by pathogens (such as but not limited to, viruses, bacteria, and fungus), cancers, and hereditary aberrations. The therapeutic agent can be, but is not limited to, DNA, plasmid DNA, RNA, protein, small molecule or combinations thereof. The composition can be made by sonication, transfection, transduction or electroporation methods. The therapeutic agent is deliverable to any specific target and Hypo-BioNV delivery can be systemic since the Hypo-BioNV was derived from an iPSC without any surface markers.

The present invention also provides for the activation of the T-cell (or other cell types) through the means of an antigen that binds to the engineered ligand (such as CAR) or through the means of an activating virus (examples as described further below) that binds to a receptor other than the engineered ligand (such as CAR), for example, a naturally occurring TCR. The activation of the T-cell (or other cell types) produces anti-cancer biomolecules such as granzymes, perforins, tumor necrosis factors, alarmins, and interleukins, to name a few. The activated T-cell, now containing the anti-cancer biomolecules is disrupted to form the Hypo-BioNVs (by any of the processes described above). The Hypo-BioNVs now contain the anti-cancer biomolecules.

The present invention also provides for Hypo-BioNVs with targeting capabilities and a method of making Hypo-BioNVs with targeting capabilities. Using standard cell line development protocols and using the CRISPR (or other gene editor)-modified allogenic iPSCs, a stable cell line can be developed where the surface marker of the desired target organ or cell type (for example ApoE for liver cell targeting), is constitutively expressed (from a strong promoter such as CMV etc.) within the CRISPR-modified allogeneic iPSC. The expression of the desired surface marker enables the iPSCs to present the surface marker on its cellular membrane. Once the surface marker is expressed on the cellular membrane of the CRISPR-modified allogeneic iPSCs (the ‘target capable’ iPSC cell line), the Hypo-BioNVs are then derived from the cell line using the sonication protocols (or any combination of the alternate methods described above) as above. The Hypo-BioNVs now have the surface marker coated on them (for example ApoE for liver cell targeting), thereby giving them specific organ targeting properties.

The present invention also provides for a composition of a therapeutic agent packaged in Hypo-BioNVs with targeting capabilities. The therapeutic agents can be packaged in the Hypo-BioNVs as described above.

The compositions herein can be used to treat any of the viruses (diseases caused by them) described above, whether lysogenic or lytic or both or diseases caused by pathogenic bacteria or fungus. The composition can also be used to treat various undesired cell types, such as pre-cancerous cells, cancer cells, or cancer cells caused by viruses.

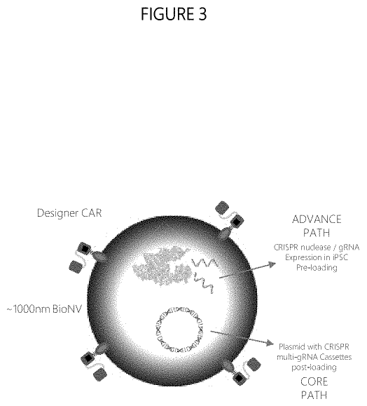

The Hypo-BioNVs can also be loaded with any CRISPR-gRNA (or gene editor) expression plasmid therapeutic for efficient and corrective gene therapy. An example is shown in FIG. 3.

CRISPR Cas9 gene editing has been used to create hypo-immunogenic hiPSC cell lines derived from human CD34+ cord blood. This CD34+ cord blood derived cell line serves as a base source for Hypo-BioNV development, production, and manufacturing for the delivery of gene editing therapeutics for hereditary disease, and anti-cancer therapeutics to the micro-environment of cancer cells. Deuse et al 2019 (8) extensively tested the CD34+ cord blood-derived hypo-immunogenic cell line to confirm the low expression of HLA 1/2 and overexpression of CD47. Once confirmed, the cells were additionally tested for their hypo-immune phenotypes in humanized mice studies. Compared to a wildtype cell line that causes INF-Y⁻ expression, an IgM reaction, and the activation of NK cells, the hypo-immunogenic iPSCs do not elicit any of these responses. Each of these experiments was duplicated (with similar results) when the iPSC line was differentiated to cardiomyocytes and epithelial cells.

CAR-T cells have been proven as an excellent source of autologous exosomes. The present invention provides the ex vivo development of allogeneic biomimetic vesicles (Hypo-BioNVs) for the delivery of gene-edited engineered CAR and other therapeutic agents without the risk of cytokine storm.

The present invention provides the ex vivo development of Hypo-BioNVs for in vivo delivery of multi-action therapeutic benefit for destroying heterogeneric cancer cells while neutralizing the immuno-suppressive properties of PD-1 and PDL-1 in the tumor microenvironment thereby enhancing the efficacy and effectiveness of the Hypo-BioNVs.

The present invention provides ex vivo development, manufacturing, purification, and delivery of CAR decorated Hypo-BioNVs that carry the genetic material to produce bi-specific antibodies that can target and block PD-1 and PDL1 (or other immune-suppressive biomolecules such as, but not limited to, DKK1) and thus more effectively fight the cancer.

The present invention provides ex vivo development, manufacturing, purification and delivery of CAR decorated Hypo-BioNVs with tunable concentrations of surface CARs and dosing.

Autologous CAR T-cells have been shown to be an excellent source of exosomes that contain CAR surface receptors (10). CAR-coated exosomes have been shown to have several key advantages over stand-alone CAR T-cell therapies. Fu et al 2019 (10) reported that autologous exosomes have potent anti-cancer properties without the occurrence of cytokine storms or runaway cytotoxicity. They have accessibility to the tumor via microenvironment without the loss of function and tumor penetration. There is zero transfer of genetic information that can lead to teratoma formation. This offers multi-target capabilities for single cancer type or multiple cancers simultaneously.

Fu et al 2019 (10) reported that autologous CAR T-cells have been shown to shed exosomes that contain equivalent concentrations of CAR receptors on their surface while containing high levels of cytotoxic molecules that inhibit tumor growth. Fu et al 2019 (10) showed that autologous CAR T-cells release about 7-8 fold higher concentrations of exosomes when they are stimulated with antigen. Immunoblot analysis showed the concentrations of CAR on the surface of autologous CAR T-cells from whole cell extracts and exosomes derived from autologous CAR T-cells stimulated with CD28/CD3 beads or cancer cell antigen stimulation. Exosomal CAR binds to cancer antigen in a concentration dependent manner and this interaction can be disrupted with blocking antibody CTX (cetuximab) and TTZ (trastuzumab). Fu et al 2019 (10). also showed that CAR exosomes have anti-tumor activity in various types of cancers. CAR-EXO-CTX (CAR exosomes with cetuximab scFv) and CAR-EXO-TTZ (CAR exosomes with trastuzumab scFv) show significant tumor reduction in mouse xenograft models containing breast cancer and lung adenocarcinoma tumors, in an increasing CAR-EXO concentration dependent manner. Patient-derived tumor tissue fragments that were established as subcutaneous xenografts were treated with 100 μg doses of CAR-EXO-TTZ show considerable tumor inhibition in HER2-positive breast and ovary cancer models.

The present invention allows for the derivation and manufacturing of biomimetic exosomes (BioNVs) from an allogenic iPSC source that can be differentiated into an activatable T-cell (or NK or macrophage, but not limited to these cell lineages). The source is not from the patient (autologous) and therefore can be used universally since it is from an engineered allogenic iPSC source. Further, the manufacturing process does not rely on naturally occurring mechanism for exosome shedding from the activated lymphocytes. Instead, the biomimetic exosomes (BioNVs) are derived from the cells through the processes described above.

In the present invention, critical gene subtractions and additions can be created ex vivo in the hypo-immunogenic iPSCs in an HLA1/HLA2 null cell line derived from CD34+ cord blood (or iPSC or stem cell source). These are also further shown in the TABLES below.

B2M−/−→HLA1 hypo-immune Hypo-1

CIITA−/−→HLA2 hypo-immune Hypo-2

CD47+/+→tgCD47 (CD47+/+ for restored phagocytosis to enhance BioNV uptake) Hypo-47

PD1−/−→PDL1 resistance elimination

Trac−/− knockout

An upstream CRISPRa CAR expression cassette with Cpf1 guided nuclease swap out system can be used to make alterations to the genes.

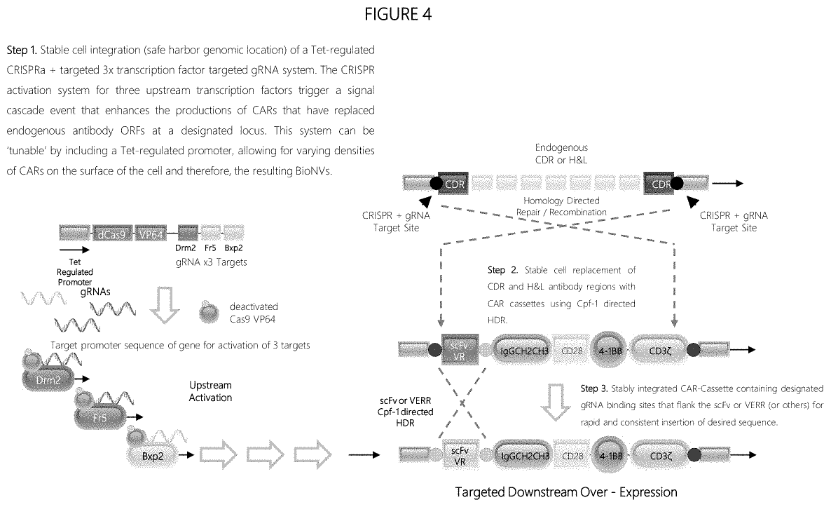

The Hypo-BioNVs of the present invention can be made as follows, and as shown in the diagram in FIG. 4. First there is stable cell integration (safe harbor genomic location, such as safe harbor sites hROSA26, AAVS1, and CCR5, as well as those listed in Pellenz, et al., Human Gene Therapy, Vol. 30 No. 7, 2019 (Site IDs 323, 325, 289, 227, 229, 253, 255, 257, 259, 261, 263, 317, 303, 331, 283, 231, 315, 327, 305, 307, 309, 285, 233, 311, 299, 301, 293, 319, 333, 295, 329, 297, 313, and 321) of a drug-regulated (such as Tet-regulated) CRISPRa+targeted 3× transcription factor targeted gRNA system (Drm2, Fr5, Bxp2—genes that have been shown to upregulate antibody production) as described above. The CRISPR activation system for three upstream transcription factors trigger a signal cascade event that enhances the productions of CARs that have replaced endogenous antibody ORFs at a designated locus. Next, there is stable replacement of CDR and H&L antibody regions with CAR cassettes using Cpf1-directed (or any other CRISPR Cas/ZFN, TALEN) HDR. Once the CAR cassette is stably integrated, the scFV, region of the cassette (near the 5′ end of the cassette) can be ‘swapped out’ for any desired scFV, VERR, V_{H}H nanobody, V_{NAR}, affilin (or other affinity targeted [bio]chemical ligands), CER, or any other single variable region of heavy chain domain using Cpf1-directed (or any other CRISPR Cas/ZFN, TALEN) HDR. The same can also be accomplished through a TCR receptor components.

The Trac locus can be knocked out from the allogenic cells so that the BioNVs do not express TCRs as well as CARs. The Trac locus can be knocked out by swapping in the CAR construct cassette in its place. In this scenario, the CAR construct cassette is not integrated in the antibody locus.

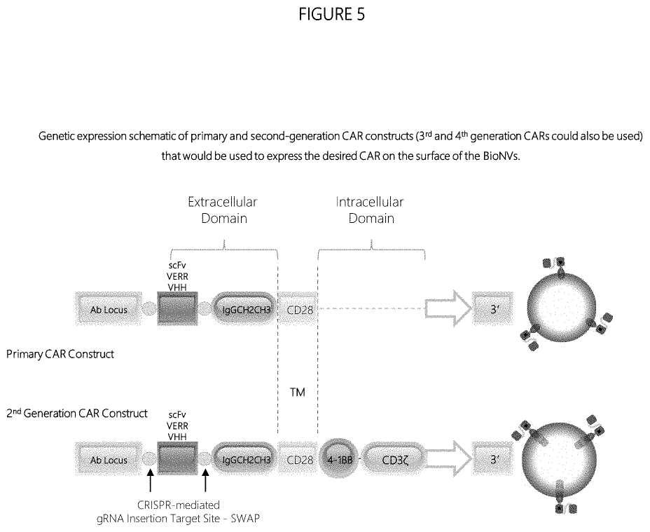

This method can be used to create Hypo-BioNVs with different functions, as shown in FIG. 5, which details the method to express first generation versus second generation CARs on the surface of the Hypo-BioNVs. For example, one type of Hypo-BioNV can be derived solely from a targeted cell line that expresses CAR (scFV, VERR, V_{H}H nanobody, V_{NAR}, affilin (or other affinity targeted [bio]chemical ligands), or CER) or TCR surface ligands only. These cell lines can be further adapted to express essential and tailored proteins or nucleic acids that can be packaged in the end-result Hypo-BioNV. Another type of Hypo-BioNV can be derived from a targeted cell line that contains the intracellular CAR (scFV, VERR, V_{H}H nanobody, V_{NAR}, affilin (or other affinity targeted [bio]chemical ligands), or CER) or TCR components necessary for the activation of lymphocytic granular and cytokine responses. Further, this foundation cell line can be differentiated from its pluripotent state into a desired lymphocyte (T-cell, NK, macrophage), from which the Hypo-BioNVs can then be derived. The resulting Hypo-BioNVs are surface coated with 2nd (or 3^{rd}) generation CAR (scFV, VERR, V_{H}H nanobody, V_{NAR}, affilin (or other affinity targeted [bio]chemical ligands), or CER) or TCR ligands and loaded with factors that elicit tumor killing.

Cell lines can be designed to express any protein and/or nucleic acid therapeutic with anti-cancer or anti-viral properties as shown in TABLE 1. Upward arrows represent an increase in expression of the genes that produce the proteins listed beside them, and downward arrows represent a decrease in expression of the genes that produce the proteins listed beside them.

TABLE 1

| Modification | Phenotype |
|---|---|
| B2M −/− CIITA −/− | HLA1/HLA2 hypo-immunity |
| CD47 +/+ | Tg CD47 |
| PD1 −/− | PDL1 resistance elimination |
| CORE Primary CAR | Specific biomarker targeting |
| Expression Cassette | |
| ↑↑ Perforin & Granzyme | Tumor cytotoxicity/apoptosis |
| B Expression | |
| Fas Ligand +ve | Induces apoptosis |
| ↑↑ NCAM (optional) | Cell adhesion (neural & hematopoietic) |
| Option Therapeutic Expression | Specific Drug Action |
| ↑↓ Differential regulation | To gauge the control of CRS |
| of Interleukins | |
| Trac −/− | Prevent cell from expressing TCR |

The present invention can also eliminate the need for the over-expression and pre-packaging of biologic therapeutics. Cell lines developed using this process contains a second-generation (or 3^{rd} generation) CAR ligand that is necessary for lymphocyte activation. Once the desired CAR ligand is expressed, the cell line is differentiated into a CAR-lymphocyte, activated with the appropriate antigen, then processed to produce ‘loaded and targeted’ Hypo-BioNVs. Cell lines can have modifications listed in TABLE 2.

TABLE 2

| Modification | Phenotype |
|---|---|
| B2M −/− CIITA −/− | HLA1/HLA2 hypo-immunity (Hypo-1, |
| | Hypo-2) |
| CD47 +/+ | Tg CD47 |
| PD1 −/− | PDL1 resistance elimination |
| ADVANCE 2^{nd}/3^{rd} Gen CAR | Specific biomarker targeting with |
| Expression Cassette | Lymphocyte Activation |
| Lymphocyte Activation | Activated Lymphocyte loaded BioNVs |
| Modifications N/A | |
| Trac −/− | Prevent cell from expressing TCR |

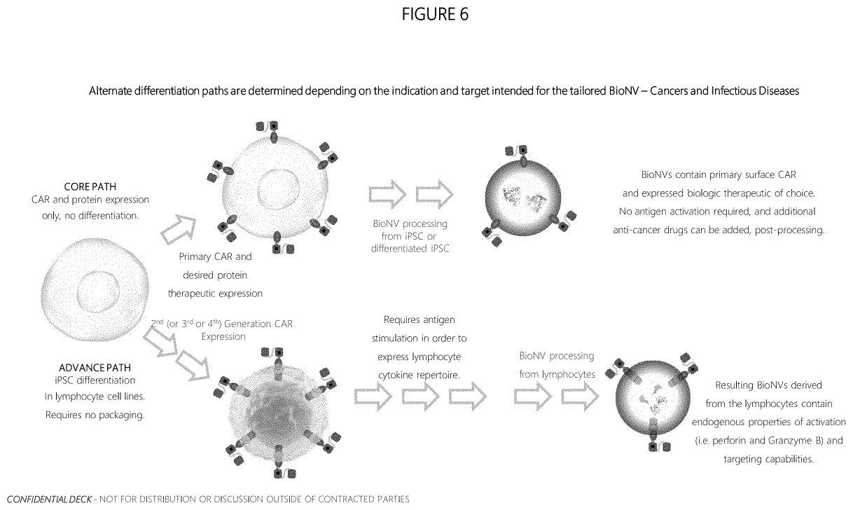

FIG. 6 shows methods of making Hypo-BioNVs in the context of primary (first-generation) versus second generation (or 3^{rd} generation) CARs on the surface of Hypo-BioNVs for delivering various types of drugs or cytokines. In one path, there is a primary (first-generation) CAR and desired protein expression (optional) only and no differentiation from the iPSC. There is primary (first-generation) CAR and desired protein therapeutic expression. There is Hypo-BioNV processing from iPSC. The resulting Hypo-BioNV contains primary (first-generation) CARs for targeting a biomarker (any type of biomarker, depending on the CAR) and can be loaded with additional drugs (small chemical compounds, peptides, or antibodies, but not limited to these molecules).

In FIG. 6, in another path, there is a second-generation (or 3^{rd} generation) CAR expressed initially on the iPSC, the iPSC is then differentiated into a lymphocyte cell (T-cell, NK, macrophage, but not limited to these cell types), the lymphocyte now contains second-generation (or 3^{rd} generation) CARs. The CAR-lymphocyte is then activated by the appropriate antigen (that recognizes the biomarker that is represented on the CAR and could be, but not limited to, an scFv, VERR, V_{H}H nanobody, V_{NAR}, affilins (or other affinity targeted [bio]chemical ligands), or CERs) or through TCR ligands and components. The antigen can be the targeted biomarker or an activating virus, or protein(s) of an activating virus (as described further below). The activation by antigen can occur by binding the antigen on a chromatography column, followed by passing the lymphocytes over the column, to be captured by the antigen. Once activated, the lymphocytes can be eluted from the column, in order to release the antigen from the CAR. The resulting eluted lymphocyte is activated. Another activation by antigen method involves the straight addition of low levels of antigen into media containing the cells, in bulk. In both cases, after activation, (through the antigen-second generation or antigen-third generation CAR interaction), the cells are treated by the methods described above to create the Hypo-BioNVs. The Hypo-BioNVs will now contain the second-generation (or 3^{rd} generation) CARs on its surface, and the essential lymphocyte activating proteins (lymphocyte anti-cancer cell or anti-defective cell cytokine repertoire), that can include, but are not limited to perforin and Granzyme B. Additional anti-cancer drugs can be added.

Activating with antigen can possibly cause difficulties because the biomarker antigen can be difficult to separate from T-cell CAR receptors. There are several viruses that when added or exposed to T-cells, activate them outside of the CAR receptor. An example of activating viruses includes (but not limited to), the two distantly related Arena Viruses; Pichinde Virus and Lymphocytic Choriomeningitis Virus. Pichinde Virus and Lymphocytic Choriomeningitis Virus have been shown to induce tumor-specific CTL responses up to 50% of the circulating CD8 T cell pool (1). Ex vivo activation of CD8 T-cells will increase the levels alarmin(s) (such as IL-1α, IL-33 and IL-17, but not limited to these interleukins), which in turn would be packaged (along with other anti-cancer biomolecules including perforins and granzymes) into the Hypo-BioNVs after their derivation from the virally activated cell. Alarmins can increase the elicit potent cytotoxic effector T lymphocyte (CTL^{eff}) responses at the site of the solid tumor. The response would be localized at the site of the tumor to where the Hypo-BioNVs that carry the alarmins are specifically targeted.

Antibodies can be attached to a piece of iron or magnetic nanoparticles (iron oxide) and magnetism can be used to separate the virus from the cells after activation, in order to clear the virus because it is desired to not transfer virus to the final preparation.

Modifications can be made as in TABLE 3. There is hypo-immunogenicity of the base iPSC, by engineering critical gene subtractions and additions into an HLA1/HLA2 null cell line derived from CD34+ cord blood or pluripotent iPSCs derived from fibroblasts or other sources.

TABLE 3

| Modification | Phenotype |
|---|---|
| B2M −/− | HLA1 hypo-immunity |
| CIITA −/− | HLA2 hypo-immunity |
| CD47 +/+ | Tg CD47 |
| PD1 −/− | PDL1 resistance elimination |
| ↑↑ NCAM (optional) | Cell adhesion (neural & hematopoietic) |
| CAR Expression Cassette | Specific biomarker targeting |
| CRISPR nuclease/gRNA | Gene Editing |
| Expression Cassette | |
| Trac −/− | Prevent cell from expressing TCR |

The present invention offers the flexibility of making several specific modifications. For example, B2M (gene ID 567) and CIITA (gene ID 4261) can be knocked out to create an allogeneic iPSC cell line, such as the cell line RCRP011N, a pluripotent iPSC from fibroblasts. There can be stable transgenic expression via lentiviral delivery of CD47 (gene ID 961) into a 2× KO (B2M, CIITA) cell line. The purpose of CD47 over-expression is to prevent phagocytosis and minimize the macrophage response. Second generation (or 3^{rd} generation) CAR cassette can be added to an Ab locus (or alternate safe harbor loci with potentially similar regulatory traits) into 2× KO. The purpose of integrating the CAR cassette into the Ab locus is to utilize the transcription factors that bind to the locus naturally to fine tune and control the expression of the CAR density on the surface of the Hypo-BioNVs. This approach allows for the increased density of the CARs on the surface of the lymphocyte prior to Hypo-BioNV derivation. High density of CAR on the surface of the Hypo-BioNVs increases the targeting efficiency of the Hypo-BioNV to its biomarker, which may exist on low concentrations in the tumor micro-environment. High density of CAR on the surface of Hypo-BioNVs is not related to CRS, an effect that occurs when the density of CAR is increased on the surface of a whole cell therapy. For example, the typical concentration range of CAR protein per microgram of T-cells is between 0.20 ng-0.70 ng. Concentrations of CAR on T-cells beyond this level can lead to increased chances of CRS when injected/infused into the body. The Hypo-BioNVs can have concentrations of CARs much higher. The Hypo-BioNVs can have CAR densities that range from 0.01 ng/μg Hypo-BioNV to saturating thresholds until a critical point where lipid density is compromised and the Hypo-BioNV ruptures. This threshold differs per CAR protein complex.

Another advantage of the tunable CAR system in the present invention is that CAR density on the Hypo-BioNV may reach a competitive threshold when targeting a biomarker. For example, too much CAR on the surface of the Hypo-BioNV may sterically inhibit the Hypo-BioNV's interaction with the intended biomarker. Therefore, just the right amount of density/concentration of CAR on the surface of the Hypo-BioNV would be desirable—The Goldilocks' Density. The tunable CAR density system is advantageous to non-tunable systems in that it: 1) Allows for controllable and optimized biomarker targeting and, 2) CRS is avoided—an issue that occurs with whole cell CAR therapies when the CAR density is too high.

In the present invention Interleukins can also be knocked out or differentially regulated in the cell prior to Hypo-BioNV derivation. Some interleukins have been shown to directly cause cytokine release syndrome (CRS). Therefore, by eliminating or differentially regulating their expression, their impact can be minimized or prevented. Some Interleukins related to CRS include IL-1, IL-4, and IL-6. Total knockouts of any one of an interleukin that causes (either upstream or downstream) CRS can be included. The Hypo-BioNVs can also include regulation (i.e., not knocked out) of an interleukin to be expressed in the range of 0%, 10%, 20%, 30%, 40%, 50%, 60%, 70%, 80%, 90%, or 100% in the case that the Interleukin has an effect to control CRS at low or high concentration (in situations where a total knockout may not be beneficial). The regulated interleukins can be regulated using a number of different approaches including replacing the wildtype promoter of each interleukin to a regulated promoter such a Tet/On or Tet/Off or others. The promoters can also be regulated directly using microRNA or CRISPRa/i based approaches. The interleukin knockouts or regulated interleukins can be done singly or in any combination (one to all) with other CRS causing (or non-CRS related) Interleukins.

In the present invention, GFP (green fluorescent protein) with a T-cell activation promoter can be knocked into 2× KO (B2M, CIITA)+1× CD47Tg. The purpose of this knock-in before the CAR cassette integration into the Ab locus is to cover all future cell lines to contain GFP, in the circumstance where GFP is allowable in the Hypo-BioNVs by regulatory bodies, but to also measure the degree of activation (which is necessary), compared to cells that do not have the knock-in (in case it is an issue with regulatory bodies).

In the present invention GFP with a T-cell activation promoter can be knocked into 3× KO (B2M, CIITA, CAR Cassette)+1× CD47Tg. The purpose of holding off on the knock-in after the CAR cassette integration (and leaving it optional to pop in on a per cassette basis) is so that cells (and the resulting Hypo-BioNVs) do not have GFP, so the Hypo-BioNVs can be used to deliver to patients without GFP in case regulatory bodies do not allow it.

GFP can be used for measuring activation of the lymphocytes or other end of the line cells used prior to deriving the Hypo-BioNVs from the cells, to know if the cells have indeed been activated. Without activation of the cells, the Hypo-BioNVs will not work. GFP can also be used as a diagnostic for tracking the Hypo-BioNVs.

Hypo-BioNVs can be made that are advantageous to use in the immunosuppressive environments of solid cancer tumors (also further described below). Such Hypo-BioNVs can include knock-ins of anti-PD-1 proteins (such as antibody or CAR recognition epitopes) to recognize PDL-1, mechanisms to reduce the adenosine in the tumor micro-environment, and increased density of CAR on the surface of the Hypo-BioNV (as mentioned above).

The present invention provides Hypo-BioNVs that can be adjusted to deliver pro-inflammatory interleukins (such as IL's 7, 12, 15, 18, and/or 23) either individually or in any combination thereof to the solid tumor environment or any other diseased cell, resulting in the recruitment of naturally occurring immune cells to the immediate and targeted vicinity (Agliardi, et al. Nat Commun. 2021 Jan. 19; 12(1):444); Ataca Atilla, et al. J Immunother Cancer. 2020 September; 8(2):e001229). The interleukins may be packaged (pre-loaded) within the Hypo-BioNVs or expressed on the surface of the Hypo-BioNVs or in a secretable form. The interleukins aid in the recruitment of, and prevent exhaustion of, naturally-occurring T-cells and/or co-therapeutic whole cell CARs. The interleukins may be pre-loaded within the Hypo-BioNVs or expressed on the surface of the Hypo-BioNVs at concentrations ranging from 0.001 ng/ug of Hypo-BioNV to saturating concentrations (defined as concentrations that do not cross the threshold of runaway inflammatory responses—CRS). The expression of the pro-inflammatory interleukins is triggered in the lymphocytes upon activation through the CAR/TCR construct. The activating domain of the CAR/TCR construct is a fourth generation CAR, also called a TRUCK. The activating domain of the TRUCK may be a 6×NFAT responsive element (such as CD3 ZAP70 cascade signaling domains) (3) with co-stimulating domains (those contained in 2^{nd} and 3^{rd} generation CARs—4-1 BB and CD3) that triggers a minimal IL-2 promoter that drives the expression of the desired and engineered interleukin (IL's 7, 12, 15, 18 and/or 23) cassette. Once the lymphocyte is activated via the CAR-to-Biomarker Immunological Synapse (IS), the engineered interleukin expression cassette is expressed the trans-protein of interest. The trans-protein can be endogenous ILs or ILs that translocate to the cellular membrane (i.e., contain and transmembrane domain). The lymphocyte is also activated in a traditional manner (as described above for 2^{nd} and 3^{rd} generations CARs). The Hypo-BioNVs are then derived from the activated TRUCK-containing (4^{th} generation CAR) lymphocyte. The resulting Hypo-BioNVs may contain the endogenous proinflammatory interleukin of choice, or any combination thereof. The Hypo-BioNVs with endogenous proinflammatory interleukins are used for systemic intravenous delivery in the body. The resulting Hypo-BioNVs may contain membrane localized proinflammatory interleukin (containing an engineered transmembrane domain) of choice, or any combination thereof. The Hypo-BioNVs with transmembrane proinflammatory interleukins are used for localized injection treatment of the solid tumor.

Other cells can be used in making the Hypo-BioNVs that have cellular distinctions. The intracellular domain from traditional CARs (4-1BB, CD3) can be replaced with activation domains from other cellular signaling moieties related to biochemical cellular functions that can be exploited for therapeutic value.

Hypo-BioNVs can be made with bispecific CARs/TCRs to recognize two biomarkers on solid tumors, virally infected cells or dysfunctional cells. Bispecific CARs/TCRs increase the targeting specificity of the Hypo-BioNVs, thereby reducing the chance of targeting healthy cells. All the applications described above (intracellular signaling moieties for 2^{nd}, 3^{rd}, and 4^{th} generation CARs and TCRs, and cytokine/chemokine regulation methods) can be adapted to the bispecific CAR/TCR targeting approach. An example of a bispecific CAR is one that includes an RVG recognition ligand.

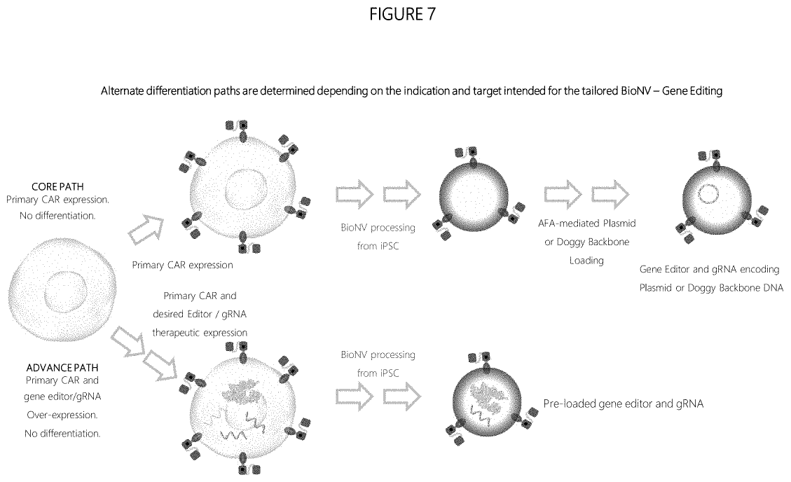

FIG. 7 shows two paths for making Hypo-BioNVs for the delivery of gene editing therapeutics. In one path, there is a primary (first-generation) CAR and desired protein expression (optional) and no differentiation. There is primary (first-generation) CAR expression. There is Hypo-BioNV processing from iPSC. There is ASA-mediated plasmid or doggy backbone loading. There is gene editor and gRNA encoding plasmid or doggy backbone DNA that is inserted into the Hypo-BioNVs via electroporation or sonication.

In another path in FIG. 7, there is primary (first-generation) CAR and gene editor/gRNA over-expression and no differentiation. There is primary (first-generation) CAR and desired editor/gRNA therapeutic expression. There is Hypo-BioNV processing from iPSC. There is pre-loaded gene editor and gRNA that is expressed from a stable integrated and drug inducible ‘gene editing cassette’ that is engineered into the iPSC.

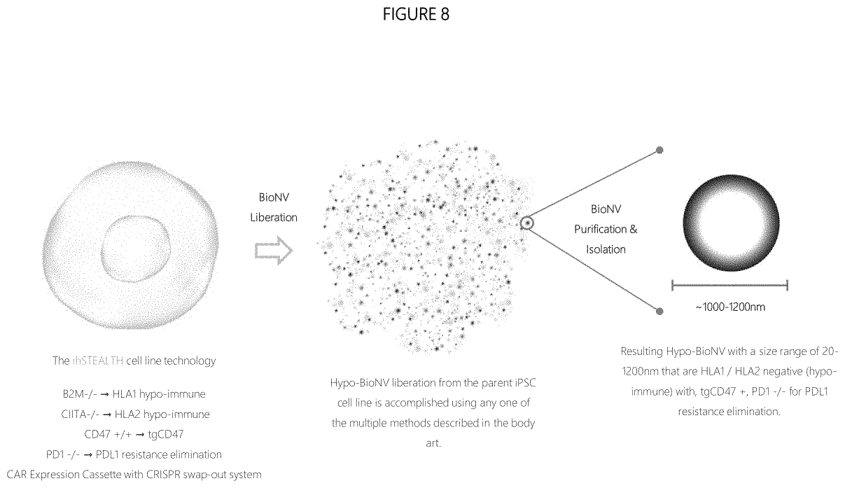

FIG. 8 shows a manufacturing process for the Hypo-BioNVs. There is Hypo-BioNV liberation from the parent iPSC cell line that is accomplished using two established methods—1) sodium deoxycholate budding (research purposes) or 2) mild detergent rupture (manufacturing). This results in Hypo-BioNVs with a size range of 1000-1200 nm that are HLA1/HLA2 negative (hypo-immune) with, CD47+/+ to promote phagocytosis, PD1−/− for PDL1 resistance elimination, although not limited to these modifications.

After serial extrusion or other manufacturing methods, there is a low probability that some of the Hypo-BioNVs can be inverted (i.e., the intercellular/intra-Hypo-BioNV signaling domains of the CAR can be flipped and pointing to the outside). This can be corrected by passing them through an affinity column containing antibodies that recognize the intracellular domains (anti-4-1BB, anti-CD3, anti-ZAP70).

The quality (specificity, safety, efficacy, and effectiveness) of the Hypo-BioNVs will be further improved by using a proprietary Artificial Intelligence platform that calculates the quality of the CAR-to-biomarker interaction, the immunological synapse (IS), that forms between the whole cell CAR and the targeted cell. Once the quality of the synapse is determined, the cells expressing the best CAR-to-biomarker target quality will be used for Hypo-BioNV derivation. The AI protocol screening method is referenced in Singh et al 2021 (29). However, since the Hypo-BioNVs are not ‘alive’ one cannot measure the immunological synapse quality directly. Measuring the synapse depends on the intercellular movement of cytokines like perforin/granzyme, F-actin etc., and these movements/localizations to the synapse are guided dynamically through cell signaling pathways. These pathways do not exist in the BioNV as the BioNV is a ‘dead’ entity with no signaling or genetic power. Therefore, when the CAR on the surface of the BioNV interacts with the targeted biomarker on a cell, the synapse does form, but the localization of all the cytokines and actins does not occur. They remain evenly dispersed throughout the BioNV. This is okay because the BioNV is so small and the issue does not translate to therapeutic ineffectiveness. The only issue, because of this, is that one cannot measure the quality of the IS interaction between the CAR and the target biomarker. So, one cannot determine the difference between a great binding CAR-to-biomarker interaction versus a poor binding CAR-to-biomarker interaction. A great binder is desired so that the BioNV delivers its therapeutic (such as anti-cancer therapeutic) to the target cell efficiently. To get around this issue the IS quality must be measured indirectly.

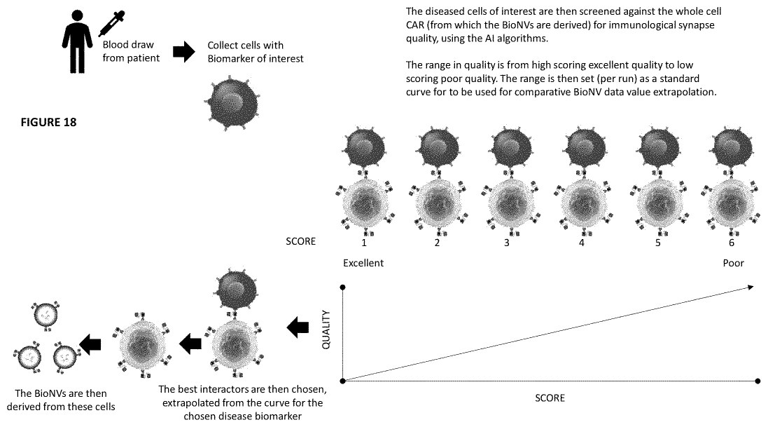

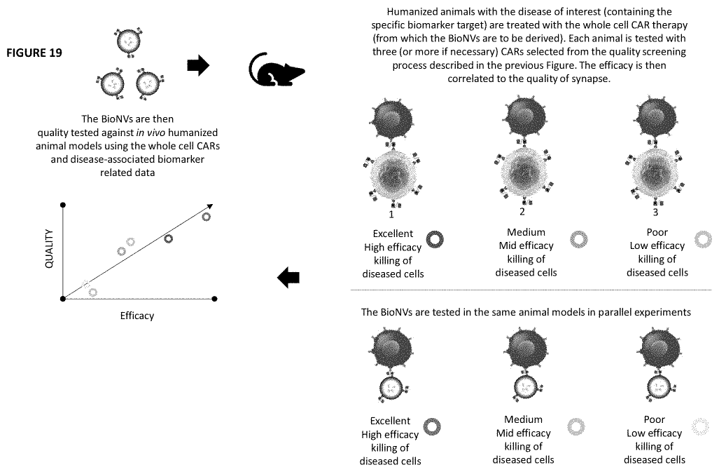

A standard curve system can be created, as shown in FIGS. 18 and 19. In FIG. 18, blood is drawn from the patient and cells are collected with a biomarker of interest. The diseased cells of interest are then screened against the whole cell CAR (from which the BioNVs are derived) for immunological synapse quality, using the AI algorithms. The range in quality is from high scoring excellent quality to low scoring poor quality. The range is then set (per run) as a standard curve for to be used for comparative BioNV data value extrapolation. The best interactors are then chosen, extrapolated from the curve for the chosen disease biomarker and the BioNVs are derived from these cells. As shown in FIG. 19, the BioNVs are then quality tested against in vivo humanized animal models using the whole cell CARs and disease-associated biomarker related data. Humanized animals with the disease of interest (containing the specific biomarker target) are treated with the whole cell CAR therapy (from which the BioNVs are to be derived). Each animal is tested with three (or more if necessary) CARs selected from the quality screening process described in FIG. 18. The efficacy is then correlated to the quality of synapse. The BioNVs are tested in the same animal models in parallel experiments.

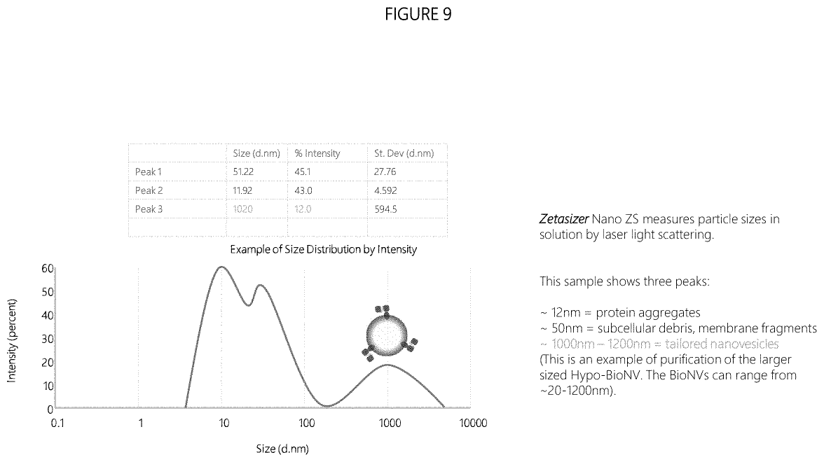

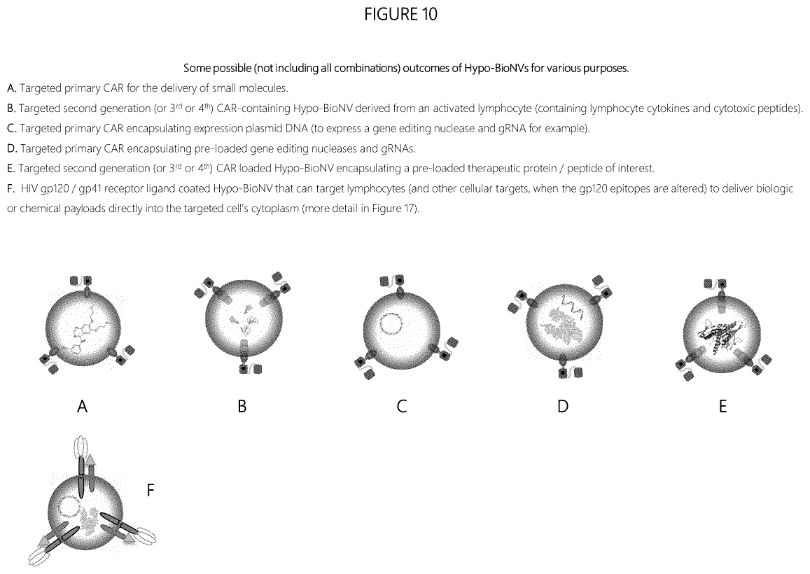

FIG. 9 shows size distribution by intensity. Zetasizer Nano ZS measured particle sizes in solution by laser light scattering. This sample shows three peaks: 1) ˜12 nm=protein aggregates, 2) ˜50 nm=subcellular debris, membrane fragments, and 3) ˜20-1000 nm=tailored nanovesicles. FIG. 10 shows examples of Hypo-BioNVs.

The Hypo-BioNVs can contain various therapeutics such as gene editors of TALENs, ZFNs, RNase P RNA, C2c1, C2c2, C2c3, Cas9, Cpf1, TevCas9, Archaea Cas9, CasY.1, CasY.2, CasY.3, CasY.4, CasY.5, CasY.6, CasX Cas omega, Saliogase, or any ortholog or homolog of any of these editors. The gene editors can also include gRNA, which, as used herein, refers to guide RNA. The gRNA can be a sequence complimentary to a coding or a non-coding sequence and can be tailored to the particular sequence to be targeted. The gRNA can be a sequence complimentary to a protein coding sequence, for example, a sequence encoding one or more viral structural proteins, (e.g., gag, pol, env and tat). The gRNA sequence can be a sense or anti-sense sequence. It should be understood that when a gene editor composition is administered herein, preferably (but not limited to) this includes two or more gRNAs; however, a single gRNA can also be used.

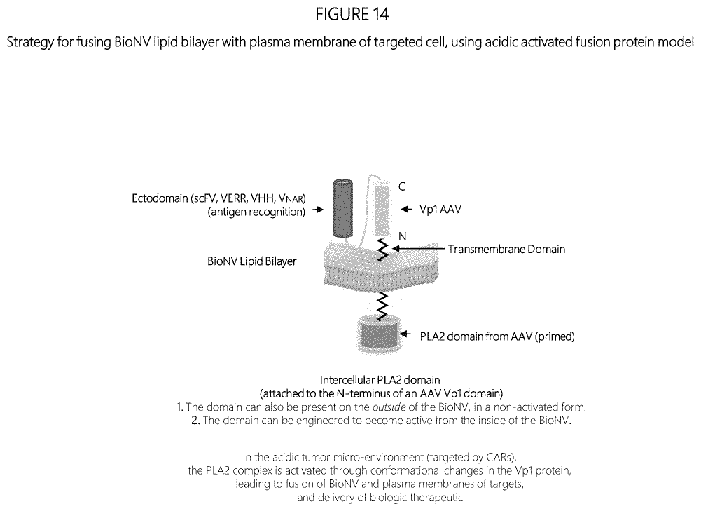

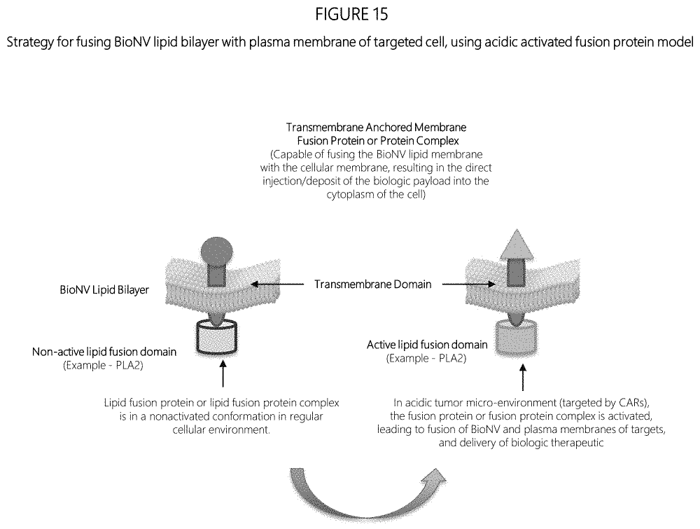

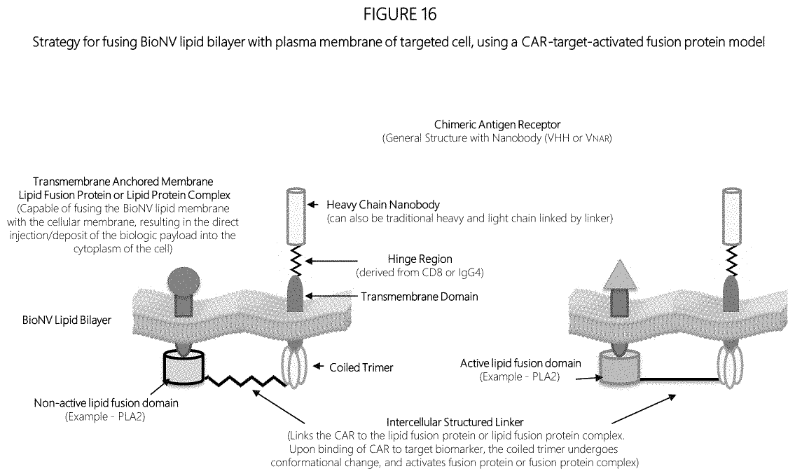

Various methods can be used to deliver nucleases, gRNA, or other therapeutic biologics directly into cells. However, one of the major issues with delivery is that once the therapeutic is taken up into the cell it generally occurs through passive diffusion (very low efficiency) or endocytosis. In the latter, the therapeutic biologic is compartmentalized in an endosome where there is a risk that it could end up being sequestered (never released) in the endosome. Further, the increasing acidic environment in the endosome, turns the endosome's properties into something resembling a lysosome, and there is an additional risk that the biologic therapeutic could be degraded/unfolded/deactivated. Viruses without envelopes (capsids) are also taken up in endosomes but have a mechanism to enter the cytoplasm during late endosomal stages. The trigger is usually the acidic environment that activates a viral fusion protein or fusion protein complex (31). For example, in AAVs the Viral protein 1 (Vp1) receptor's N-terminus is tethered to an enzymatic domain that has phospholipase A2 (PLA2) activity (38). When the Vp1 N-terminus with the PLA domain is exposed to the slightly acidic endosome environment (pH ˜6.0), it is triggered and facilitates the escape of the virus into cytoplasm by rupturing the endosome membrane (30). Alternately, viruses with envelopes do not enter the host cell through an endocytosis mechanism but have protein fusion mechanisms that allow for the direct fusion of their lipid membranes to the host cellular membrane resulting in the release of the viral contents directly into the cytoplasm. Both capsid and/or envelope-based viral-cellular entry mechanisms can be exploited, in order to ensure effective and active delivery of biologic therapeutics using Hypo-BioNVs. By mimicking viral fusion mechanisms associated with 1) some non-enveloped capsid viruses, such as the engineering the viral proteins like viral PLA2 from Vp1 of AAV to be expressed on the surface of a Hypo-BioNV, one can facilitate the exit of a biologic therapeutic from Hypo-BioNVs encapsulated in endosomes into the cytoplasm (FIGS. 14-16). Further, by mimicking viral fusion mechanisms associated with 2) enveloped proteins, such as gp120/gp41 of HIV (although a complex mechanism, the gp41 protein could be engineered to harpoon the lipid membrane of the target cell), one can facilitate the exit of a biologic from a Hypo-BioNV directly into the cytoplasm of its targeted cell, at the plasma membrane interface. Each approach/method (capsid or envelope-based properties) can be engineered using artificial intelligence design.

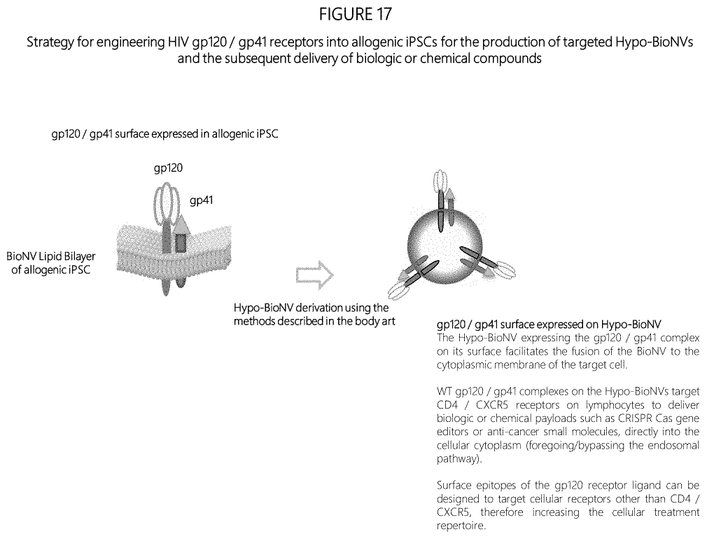

Expanding on the above concept of gp120/gp41 receptor ligand/fusion protein complexes that are embedded in the Hypo-BioNV membrane to facilitate target cell membrane fusion and subsequent release of the Hypo-BioNVs payload (any biologic, nucleic acid, peptide, or small molecule, but not limited to these), the gp120 and gp41 can be engineered, through artificial intelligence algorithms, to recognize alternate targets other than CD4+ receptors and penetrate the cell membrane (FIG. 17). In particular, the gp120 variable regions that recognize the CD4+ receptor can be altered to epitopically recognize non-CD4+ biomarkers on cancer cells and low-no affinity to CD4, cells infected with infective agents including viruses, bacteria, or fungus, cells with genetic dispositions related to hereditary diseases, cells expressing autoimmune markers and cells that are dysfunctional and related/causative of metabolic disorders. In addition to the engineered gp120 recognition sequences, the gp120 protein can be swapped with versions that have been identified to be less immunogenic than others, based on an HIV mechanism to avoid host immune reactions (33), with the purpose increase the probability of multiple dosing and avoid the build-up of immunity against a single type of protein receptor complex. Gp41 can be swapped in combination in a similar manner as well. AI algorithms can be combined with Deepmind to find multiple gp120/gp41 complexes from the vast HIV mutant flora that have low immunogenicity. This allows for the design mini-VAN BioNVs that can be used in previously exposed HIV patients or patients (not HIV exposed) with other diseases that affect white blood cells. The complexes can be rotated for multiple injections if necessary. The second generation of mini-VAN BioNVs can have gp120/gp41 complexes that have been designed to recognize biomarker targets other than CD4/CCR5 receptor complexes on white blood cells.

The Hypo-BioNVs can alternatively include the cell-penetrating peptide of RAAS from CD2v embedded in the membrane that can pierce a cell membrane, facilitate membrane fusion, and deliver the payload to the cytoplasm of the target cell. CD2v is a transmembrane protein encoded by the EP402R gene of the African Swine Fever Virus (ASFV). It contains repetitive amino acid sequences ([KPCPPP]₃ labeled as RAAS), which is reported as a genetic marker and an epitope. The RAAS of CD2v can act as a cell-penetrating peptide (Yang, et al., Drug Deliv. 2021 December; 28(1):957-962.)

In the present invention, other fusion peptides that could be mimicked and engineered (using artificial intelligence design) into the surface of a Hypo-BioNV include fusogens, and viral FAST proteins to achieve the delivery of biomolecules (such as gene editors) directly into the target cell(s) at the cytoplasmic membrane.

In another approach the mechanisms of tSNARES and vSNARES can be exploited as well as peptides similar to FAST proteins or any other suitable peptide can be created with an AI platform to enhance delivery.

In another approach, the surface lipid bilayer of the Hypo-BioNVs can be charged with positively charged lipids and/or transmembrane integrated cationic peptides (the latter of appropriate density to confer specificity to endosomes and not other bilayer membranes or compartments) that create a charge differential in an endosome environment, thereby disrupting the endosome resulting in the exit of the biologic therapeutic into the cytoplasm of the target cell (12).

The present invention provides for a method of treating an individual with cancer, an infectious disease, or hereditary disease, by administering the Hypo-BioNVs to an individual, targeting: 1) cancer cells, 2) cells that have been biochemically or genetically corrupted by (but not limited to) an infectious pathogen such as a virus, bacteria, or fungus, or 3) cells that have hereditary aberrations or genetic mutations, and treating the cancer, infectious disease, or hereditary disease. The CAR receptor (that may consist of either an scFV, VERR, V_{H}H nanobody, V_{NAR}, affilins (or other affinity targeted [bio]chemical ligands), or CERs) or TCR ligand/components can recognize its specific biomarker on the cancer cell of a tumor, stem-like cancer cells (circulating tumor cells) that shed from the tumor, endothelial cells that make up the neovascular region surrounding the tumor, and cancer cells that exist within the stroma. Other cells can be targeted with infectious/hereditary diseases. Once the CAR docks/interacts with the biomarker on the cancer or other cell (the target), it releases its payload (drug, cytokine, peptide, gene editor/gRNA, plasmid etc.)

Examples (but not limited to) of current whole cell therapies that can be adapted to the Hypo-BioNV CAR methodology are shown in TABLE 4.

TABLE 4

| | Examples of Current Whole Cell | Typical BioNV genetic |
|---|---|---|
| Indications | Approach to be Adopted to BioNVs | composition |
| HPV associated | HPV16-E7 targeted TCR-T cells for HLA- | Allogenic iPS-derived and |
| tumors (Cervical, | A*02:01-positive patients (KITE-439, | genetically modified hypo- |
| head and neck, | NCT02858310, NCT04015336, | immune response, no-to-low |
| anogenital, and other) | NCT04044950) | cytokine storm and other |
| | T1E28ζ-ErbB ligand-CD28+CD3ζ plus | toxicities, off-the-shelf, |
| | 4αβ, an IL-4-responsive CAR-T cells (T4 | consistent, robust and |
| | CAR-T, NCT01818323) | scalable process and low cost |
| | Lenti-2G-Muc1-CAR-T-2A-IL22 (secreted | of goods, targeting to one or a |
| | form of IL22). HLA-DPB1*04:01 Positive | combination of tumor markers |
| | (Mei et al. 2019 (A)) | listed in the middle column, |
| | MAGE-A3/A6-tageted TCR-T cells (Kite- | incorporating anti-checkpoint |
| | 718, NCT03139370) | inhibition (e.g., PD-1 KO) and |
| | Other targets Mesothelin, PSMA, GD2, | metabolic and inflammatory |
| | Her2, αvβ6, L1-CAM | genetic elements or respective |
| Glioma and | CAR-T cells with or without PD-1 KO | proteins (e.g., IL12, IL15, IL18, |
| Glioblastoma | targeting to tumor markers EGFRvIII, | IL7R) encapsulated. Can be |
| | EGFR806, GD2, Muc1, Her2, EphA2, B7- | delivered as CAR-T, TCR-T, |
| | H3, CAIX, αvβ3, IL13Ra2, CD56, Glypican | CAR-NK and other cellular |
| | 2, CD171 (NCT03500991, NCT03638167, | therapies. |
| | NCT01454596, NCT02208362, | Delivered parenteral, |
| | NCT02575261, NCT04099797) | intratumor or regional. |
| Ocular Melanoma | TCR-T cells targeting gp100 (Gp100 fused | |
| (majority are uveal | to an anti-CD3 scFv) for HLA A2 positive | |
| melanoma) | patients (Tebentafusp (IMCgp100)) for | |
| | Uveal melanoma (NCT03070392) | |
| | GD2-IL15-CAR-T (Wang et al. 2020 (B)) | |
| Pancreatic | EpCam-CD3 zeta and CD28 CAR-T cells | |
| (Pancreatic ductal | (NCT03013712) | |
| adenocarcinoma) | Anti-CEA CAR-T Cells (NCT04037241) | |
| | TAA-specific cytotoxic T lymphocytes: NY- | |
| | ESO-1, MAGEA4, PRAME, Survivin or | |
| | SSX2 (NCT03192462) | |
| | CART-meso (NCT03323944, | |
| | NCT03638193) | |
| | Targeting to other tumor markers PSCA, | |
| | CEACAM7 (CGM2), Her2, Claudin 18.2, | |
| | CD70, CD133, B7-H3. CD133 | |
| Breast Cancers | CAR-T cells targeting | Allogenic iPS-derived and |
| | Mesothelin (NCT02792114) | genetically modified hypo- |
| | Mucin1 (Anti-MUC1* scFv-CD8-4-1BB- | immune response, no-to-low |
| | CD3-z, NCT04020575) | cytokine storm and other |
| | Her2 (HER2(EQ) BBzeta/CD19t+, | toxicities, off-the-shelf, |
| | NCT03696030) | consistent, robust and |
| | HER-2, GD2, CD44v6 simultaneously | manufacturing process and |
| | (NCT04430595) | consequent low cost of goods, |
| | EpCAM (NCT02915445) | targeting to one or a |
| | Others: CD70, ROR1, CEA, FR. CD7 | combination of tumor markers |
| Ovarian, fallopian, | Mucin-targeted | listed in the middle column, |
| peritoneal | Mucin16 targeted + membrane bound IL- | incorporating anti-checkpoint |
| | 15 (NCT03907527) | inhibition (e.g., PD-1 KO) and |
| | Mesothelin-targeted CAR-T | metabolic and inflammatory |
| | NCT03916679, NCT04562298, | genetic elements or respective |
| | NCT03814447, NCT03608618, | proteins (e.g., IL12, IL15, IL18, |
| | NCT02159716, NCT03054298 | IL7R) encapsulated. Can be |
| | Anti-ALPP (NCT04627740) | delivered as CAR-T, TCR-T, |
| | +Secreting PD-1 | CAR-NK and other cellular |
| | Nanobodies (NCT04503980) | therapies. |
| | Mesothelin-targeted CAR-NK | Delivered parenteral, |
| | Anti-ALPP (NCT03692637 | intratumor or regional. |
| | Her2-targeted CAR-T, | |
| | NCT04511871 | |
| | Alpha Folate | |
| | Receptor-targeted CAR-T NCT03585764 | |
| | Multiple target CAR-T cells | |
| | NCT03638206 | |
| | Other targets: anti-CD19, | |
| | CD22, CD33, BCMA, CD38, NY | |
| | ESO-1 DR5, EGFRvIII, Mucin1, | |
| | CD70, CD133, αvβ6, B7-H3 | |
| | CD47, NKG2D, CEA | |
| Lung Cancers | Mucin1-targeted CAR-T | Allogenic iPS-derived and |
| | +anti-CTLA-4 and PD-1 NCT03179007 | genetically modified hypo- |
| | NCT03525782 | immune response, no-to-low |
| | +/−PD-1 KO, NCT03525782 | cytokine storm and other |
| | CD137/CD28-CD3) zeta-PD-L1 | toxicities, off-the-shelf, |
| | scFv CAR-T NCT03330834 | consistent, robust and |
| | EGFR-CXCR5-CAR-T cells | scalable manufacturing |
| | NCT04153799 | process and consequent low |
| | Multi-target CAR-T | cost of goods, targeting to one |
| | NCT03198052 | or a combination of tumor |
| | GPC3-CART cell and/or soluble | markers listed in the middle |
| | GPC3/TGFβ-CART | column, incorporating anti- |
| | NCT03198546 | checkpoint inhibition (e.g., PD- |
| | Single or multi-targeted CAR- | 1 KO) and metabolic and |
| | TNCT03356808, NCT03535246 | inflammatory genetic elements |
| | Other targets: mesothelin, Her2. CD70, | or respective proteins (e.g., |
| | CD133, CD32A, ROR, EGFRvIII, FAP, | IL12, IL15, IL18, IL7R) |
| | CEA, PSCA, MAGE-A1/3/4, GD2, Lewis-Y, | encapsulated. Can be |
| | AXL, EGFR, B7-H3, Claudin18.2 | delivered as CAR-T, TCR-T, |
| Multiple Solid Tumors | CAR-NK cells | CAR-NK and other cellular |
| | FT536, Allogenic, iPS derived, | therapies. |
| | CAR-MICA/B-hnCD16-IL15RF- | Delivered parenteral, |
| | CD38-KO | intratumor or regional. |
| | FT573, Allogenic, iPS-derive, | |
| | dhnCD16 + IL15RF + CD38-KO + | |
| | CAR-target B7H3 | |
| | CAR-T cells | |
| | CTX110, allogenic, CD19, TCR | |
| | KO, MHC KO, NCT04438083 | |
| | BPX-601: Anti-PSCA-CD3ζ CAR- | |
| | MyD88/CD40 domain, | |
| | NCT02744287 | |
| | Targeting mesothelin, | |
| | NCT03054298, NCT02159716, | |
| | NCT02414269 | |
| | Targeting Her2 | |
| | BPX-603, Anti-Her2-Inducible co-activation | |
| | domain MyD88/CD40, NCT04650451, | |
| | NCT01935843 | |
| | EGFR806-EGFRt and 4 1 BBζ CD19- | |
| | Her2tG NCT03618381 | |
| | NCT03740256 | |
| | Targeting to CD133, NCT02541370 | |
| | Directed to Claudin 18.2, | |
| | NCT03874897, NCT04404595 | |
| | Directed to TnMUCI + TGFβDN | |
| | receptor NCT04025216 | |
| | Directed to CD44v6, | |
| | NCT04427449 | |
| | TCR-T cells: HLA-A*02 specific | |
| | MAGEA4/8, NCT03054298 | |
| | MAGEA1, NCT03441100 | |
| | PRAMA, NCT03686124 | |
| | MAGE A3, A6 KITE-718, | |
| | NCT03139370 | |
| | Mesothelin-targeted | |
| | NCT03907852 | |
| | Personalized TCR | |
| | NCT03891706 | |
| | Ant-CD19-membrane-bound | |
| | IL-15 (MD Anderson) | |

In the present invention, the Hypo-BioNVs can target cancer cells associated with adenoid cystic carcinoma, adrenal gland tumors, amyloidosis, anal cancer, appendix cancer, astrocytoma, ataxia-telangiectasia, attenuated familial adenomatous polyposis, Beckwith-Wiedermann Syndrome, bile duct cancer, Birt-Hogg-Dube Syndrome, bladder cancer, bone cancer, brain stem glioma, brain tumors, breast cancer, carcinoid tumors, Carney complex, central nervous system tumors, cervical cancer, colorectal cancer, Cowden syndrome, craniopharyngioma, desmoplastic infantile ganglioglioma, endocrine tumors, ependymoma, esophageal cancer, Ewing sarcoma, eye cancer, eyelid cancer, fallopian tube cancer, familial adenomatous polyposis, familial malignant melanoma, familial non-VHL clear cell renal cell carcinoma, gallbladder cancer, Gardner Syndrome, gastrointestinal stromal tumor, germ cell tumor, gestational trophoblastic disease, head and neck cancer, diffuse gastric cancer, leiomyomatosis and renal cell cancer, mixed polyposis syndrome, pancreatitis, papillary renal cell carcinoma, HIV and AIDS-related cancer, islet cell tumors, juvenile polyposis syndrome, kidney cancer, lacrimal gland tumor, laryngeal and hypopharyngeal cancer, acute lymphoblastic leukemia, acute lymphocytic leukemia, acute myeloid leukemia, B-cell prolymphocytic leukemia, hairy cell leukemia, chronic lymphocytic leukemia, chronic myeloid leukemia, chronic T-cell lymphocytic leukemia, eosinophilic leukemia, Li-Fraumeni Syndrome, liver cancer, lung cancer, Hodgkin lymphoma, Non-Hodgkin lymphoma, Lynch Syndrome, mastocytosis, medulloblastoma, melanoma, meningioma, mesothelioma, Muir-Torre Syndrome, multiple endocrine neoplasia type 1, multiple endocrine neoplasia type 2, multiple myeloma, myelodysplastic syndromes, MYH-associated polyposis, nasal cavity and paranasal sinus cancer, nasopharyngeal cancer, neuroblastoma, neuroendocrine tumors, neurofibromatosis type 1, neurofibromatosis type 2, nevoid basal cell carcinoma syndrome, oral and oropharyngeal cancer, osteosarcoma, ovarian cancer, pancreatic cancer, parathyroid cancer, penile cancer, Peutz-Jeghers Syndrome, pituitary gland tumors, pleuropulmonary blastoma, prostate cancer, retinoblastoma, rhabdomyosarcoma, salivary gland cancer, sarcoma, alveolar soft part and cardiac sarcoma, Kaposi sarcoma, skin cancer, small bowel cancer, stomach cancer, testicular cancer, thymoma, thyroid cancer, tuberous sclerosis syndrome, Turcot Syndrome, unknown primary, uterine cancer, vaginal cancer, Von Hippel-Lindau Syndrome, Wilms tumors, or Xeroderma pigmentosum.

In another embodiment the Hypo-BioNVs can target any cells associated with infectious diseases, such as viral, protozoan, or bacterial disease not limited to influenza, measles, COVID-19, AIDS, amebiasis, anaplasmosis, anthrax, antibiotic resistance, avian influenza, babesiosis, botulism, brucellosis, campylobacter, cat scratch disease, chickenpox, chikungunya, Chlamydia trachomatis, cholera, Clostridium perfringens, conjunctivitis, crusted scabies, cryptosporidiosis, cyclospora, dengue fever, diphtheria, ebola virus disease, E. coli, eastern equine encephalitis (EEE), enterovirus 68, fifth disease, genital herpes, genital warts, giardia, gonorrhea, group A Streptococcus, Guillain-Barré syndrome, Hand, Foot & Mouth Disease, Hansen's disease, hantavirus, lice, hepatitis A, hepatitis B, hepatitis C, herpes, herpes B virus, Hib disease, histoplasmosis, HIV, HPV (Human Papillomavirus), impetigo, Kawasaki syndrome, legionellosis, leprosy, leptospirosis, listeriosis, lyme disease, lymphocytic choriomeningitis (LCMV), malaria, Marburg virus, meningitis, meningococcal disease, MERS (Middle East Respiratory Illness), monkeypox, mononucleosis, MRSA, mumps, Mycoplasma pneumoniae, neisseria meningitis, norovirus, Orf Virus (Sore Mouth), pelvic inflammatory disease (PID), PEP, pertussis, pink eye, plague, pneumococcal disease, powassan virus, psittacosis, Q fever, rabies, raccoon roundworm, rat bite fever, Reye's Syndrome, Rickettsialpox, ringworm, rubella, salmonella, scabies, scarlet fever, shigella, shingles, smallpox, strep throat, syphilis, tetanus, toxoplasmosis, trichinosis, trichomoniasis, tuberculosis, tularemia, varicella, vibriosis, viral hemorrhagic fevers (VHF), West Nile virus, whooping cough, yellow fever, yersiniosis, or zika virus.

In the present invention, the several cancer biomarkers can be targeted including but are not limited to, Mesothelin, ER, PR, HER-2/neu, EGFR, KRAS, UGT1A1, c-KIT, CD20, CD30, PDGFR, TEM8, EIIIB, or CA-125.

The CD147 biomarker for SARS Cov-2 can also be targeted via CAR (which may contain an scFV, VERR, V_{H}H nanobody, V_{NAR}, affilin (or other affinity targeted [bio]chemical ligands), or CER) or TCR ligand recognition on the surface of Hypo-BioNVs to treat cells infected with SARS Cov-2.

The CD147 targeted biomarker can be used as a co-therapeutic in combination with CAR-NK CD147 cells (reference WO2020190483A1) to treat SARS Cov2 infected cells.

The Hypo-BioNVs can target any cells associated with hereditary diseases such as, but not limited to, 1p36 deletion syndrome, 18p deletion syndrome, 21-hydroxylase deficiency, Alpha 1-antitrypsin deficiency, AAA syndrome (achalasia-addisonianism-alacrima syndrome), Aarskog-Scott syndrome, ABCD syndrome, Aceruloplasminemia, Acheiropodia, Achondrogenesis type II, achondroplasia, Acute intermittent porphyria, adenylosuccinate lyase deficiency, Adrenoleukodystrophy, Alagille syndrome, ADULT syndrome, Aicardi-Goutières syndrome, Albinism, Alexander disease, alkaptonuria, Alport syndrome, Alternating hemiplegia of childhood, Amyotrophic lateral sclerosis-Frontotemporal dementia, Alström syndrome, Alzheimer's disease, Amelogenesis imperfecta, Aminolevulinic acid dehydratase deficiency porphyria, Androgen insensitivity syndrome, Angelman syndrome, Apert syndrome, Arthrogryposis-renal dysfunction-cholestasis syndrome, Ataxia telangiectasia, Axenfeld syndrome, Beare-Stevenson cutis gyrata syndrome, Beckwith-Wiedemann syndrome, Benjamin syndrome, biotinidase deficiency, Bjornstad syndrome, Bloom syndrome, Birt-Hogg-Dube syndrome, Brody myopathy, Brunner syndrome, CADASIL syndrome, CRASIL syndrome, Chronic granulomatous disorder, Campomelic dysplasia, Canavan disease, Carpenter Syndrome, Cerebral dysgenesis-neuropathy-ichthyosis-keratoderma syndrome (SEDNIK), Cystic fibrosis, Charcot-Marie-Tooth disease, CHARGE syndrome, Chediak-Higashi syndrome, Cleidocranial dysostosis, Cockayne syndrome, Coffin-Lowry syndrome, Cohen syndrome, collagenopathy, types II and XI, Congenital insensitivity to pain with anhidrosis (CIPA), Congenital Muscular Dystrophy, Cornelia de Lange syndrome (CDLS), Cowden syndrome, CPO deficiency (coproporphyria), Cranio-lenticulo-sutural dysplasia, Cri du chat, Crohn's disease, Crouzon syndrome, Crouzonodermoskeletal syndrome (Crouzon syndrome with acanthosis nigricans), Darier's disease, Dent's disease (Genetic hypercalciuria), Denys-Drash syndrome, De Grouchy syndrome, Down Syndrome, Di George's syndrome, Distal hereditary motor neuropathies, Distal muscular dystrophy, Duchenne muscular dystrophy, Dravet syndrome, Edwards Syndrome, Ehlers-Danlos syndrome, Emery-Dreifuss syndrome, Epidermolysis bullosa, Erythropoietic protoporphyria, Fanconi anemia (FA), Fabry disease, Factor V Leiden thrombophilia, Fatal familial insomnia, Familial adenomatous polyposis, Familial dysautonomia, Familial Creutzfeld-Jakob Disease, Feingold syndrome, FG syndrome, Fragile X syndrome, Friedreich's ataxia, G6PD deficiency, Galactosemia, Gaucher disease, Gerstmann-Sträussler-Scheinker syndrome, Gillespie syndrome, Glutaric aciduria, type I and type 2, GRACILE syndrome, Griscelli syndrome, Hailey-Hailey disease, Harlequin type ichthyosis, Hemochromatosis, hereditary, Hemophilia, Hepatoerythropoietic porphyria, Hereditary coproporphyria, Hereditary hemorrhagic telangiectasia (Osler-Weber-Rendu syndrome), Hereditary inclusion body myopathy, Hereditary multiple exostoses, Hereditary spastic paraplegia (infantile-onset ascending hereditary spastic paralysis), Hermansky-Pudlak syndrome, Hereditary neuropathy with liability to pressure palsies, Heterotaxy, Homocystinuria, Huntington's disease, Hunter syndrome, Hurler syndrome, Hutchinson-Gilford progeria syndrome, Hyperlysinemia, Hyperoxaluria, primary, Hyperphenylalaninemia, Hypoalphalipoproteinemia (Tangier disease), Hypochondrogenesis, Hypochondroplasia, Immunodeficiency-centromeric instability-facial anomalies syndrome (ICF syndrome), Incontinentia pigmenti, Ischiopatellar dysplasia, Isodicentric, Jackson-Weiss syndrome, Joubert syndrome, Juvenile primary lateral sclerosis (JPLS), Keloid disorder, Kniest dysplasia, Kosaki overgrowth syndrome, Krabbe disease, Kufor-Rakeb syndrome, LCAT deficiency, Lesch-Nyhan syndrome, Li-Fraumeni syndrome, Limb-Girdle Muscular Dystrophy, Lynch syndrome, lipoprotein lipase deficiency, Malignant hyperthermia, Maple syrup urine disease, Marfan syndrome, Maroteaux-Lamy syndrome, McCune-Albright syndrome, McLeod syndrome, MEDNIK syndrome, Mediterranean fever, Menkes disease, Methemoglobinemia, Methylmalonic acidemia, Micro syndrome, Microcephaly, Morquio syndrome, Mowat-Wilson syndrome, Muenke syndrome, Multiple endocrine neoplasia type 1 (Wermer's syndrome), Multiple endocrine neoplasia type 2, Muscular dystrophy, Muscular dystrophy (Duchenne and Becker type), Myostatin-related muscle hypertrophy, myotonic dystrophy, Natowicz syndrome, Neurofibromatosis type I, Neurofibromatosis type II, Niemann-Pick disease, Nonketotic hyperglycinemia, Nonsyndromic deafness, Noonan syndrome, Norman-Roberts syndrome, Ogden syndrome, Omenn syndrome, Osteogenesis imperfecta, Pantothenate kinase-associated neurodegeneration, Patau syndrome (Trisomy 13), PCC deficiency (propionic acidemia), Porphyria cutanea tarda (PCT), Pendred syndrome, Peutz-Jeghers syndrome, Pfeiffer syndrome, Phenylketonuria, Pipecolic acidemia, Pitt-Hopkins syndrome, Polycystic kidney disease, Polycystic ovary syndrome (PCOS), Porphyria, Prader-Willi syndrome, Primary ciliary dyskinesia (PCD), Primary pulmonary hypertension, Protein C deficiency, Protein S deficiency, Pseudo-Gaucher disease, Pseudoxanthoma elasticum, Retinitis pigmentosa, Rett syndrome, Roberts syndrome, Rubinstein-Taybi syndrome (RSTS), Sandhoff disease, Sanfilippo syndrome, Schwartz-Jampel syndrome, Sjogren-Larsson syndrome, Spondyloepiphyseal dysplasia congenita (SED), Shprintzen-Goldberg syndrome, Sickle cell anemia, Siderius X-linked mental retardation syndrome, Sideroblastic anemia, Sly syndrome, Smith-Lemli-Opitz syndrome, Smith-Magenis syndrome, Snyder-Robinson syndrome, Spinal muscular atrophy, Spinocerebellar ataxia (types 1-29), SSB syndrome (SADDAN), Stargardt disease (macular degeneration), Stickler syndrome, Strudwick syndrome (spondyloepimetaphyseal dysplasia, Strudwick type), Tay-Sachs disease, Tetrahydrobiopterin deficiency, Thanatophoric dysplasia, Treacher Collins syndrome, Tuberous sclerosis complex, Turner syndrome, Usher syndrome, Variegate porphyria, von Hippel-Lindau disease, Waardenburg syndrome, Weissenbacher-ZweymUller syndrome, Williams syndrome, Wilson disease, Woodhouse-Sakati syndrome, Wolf-Hirschhorn syndrome, Xeroderma pigmentosum, X-linked intellectual disability and macroorchidism (fragile X syndrome), X-linked spinal-bulbar muscle atrophy (spinal and bulbar muscular atrophy), Xp11.2 duplication syndrome, X-linked severe combined immunodeficiency (X-SCID), X-linked sideroblastic anemia (XLSA), 47,XXX (triple X syndrome), XXXX syndrome (48, XXXX), XXXXX syndrome (49, XXXXX), XYY syndrome (47,XYY), or Zellweger syndrome.

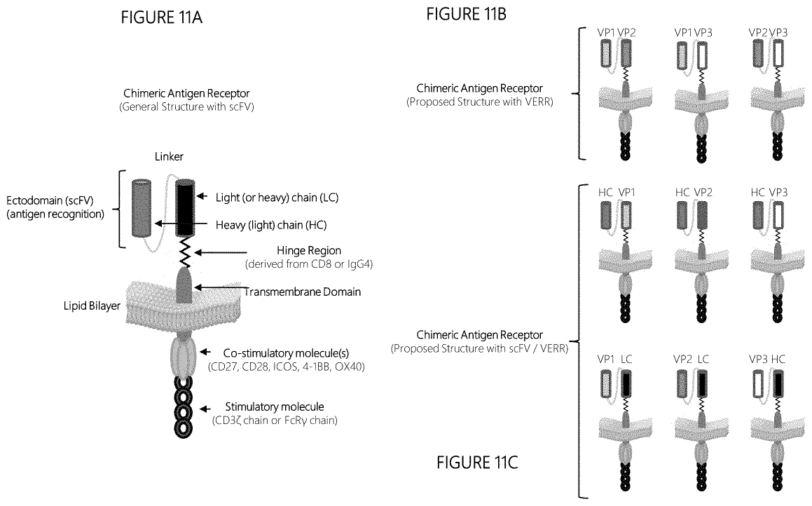

More specifically, oncolytic viruses (the receptors) can be used in combination with the Hypo-BioNVs for treating cancer, hereditary, infectious diseases, autoimmune disease, or metabolic disorders. Oncolytic viruses have the ability to target cancer cells (and others) and deliver anti-cancer medicines when they are deactivated. FIG. 11A shows a representation of the general structure of a CAR receptor with scFV. The scFV region of the CAR receptor can be replaced with a VERR that includes viral receptors of oncolytic viruses. For example, the VP1, VP2 or VP3 (or partial segments of these viral protein receptors that contain the viral target recognition epitope) of Seneca Valley virus (SVV) can be used to replace the scFV in a CAR receptor in any combination (joined by a linker) such as VP1/VP2 or VP2/VP3 or VP1/VP3, shown in FIG. 11B. Also, each Hypo-BioNV can be ‘decorated’ with any combination of these VERRs simultaneously, to increase the probability of interacting with its target (in the case of SVV, the target is TEM8). The oncolytic viruses can be, but are not limited to, vaccinia virus, vesicular stomatitis virus, poliovirus, reovirus, Seneca Valley virus, Semliki Forest virus (SFV), maraba virus, or coxsackievirus. Therefore, the present invention provides for Hypo-BioNVs including tailored CARs which can recognize target biomarkers through a VERR including viral receptors of an oncolytic virus.

This approach is advantageous over the use of ‘gutted’ deactivated viruses for the following reasons. The BioNVs are derived from hypoimmunogenic cells, therefore immune reaction is vastly minimized compared to viruses. There is no chance for infection. The Hypo-BioNVs can carry bigger payloads (as most oncolytic viruses are small) such as gene editors (proteins), nucleic acids or higher concentrations of drugs.

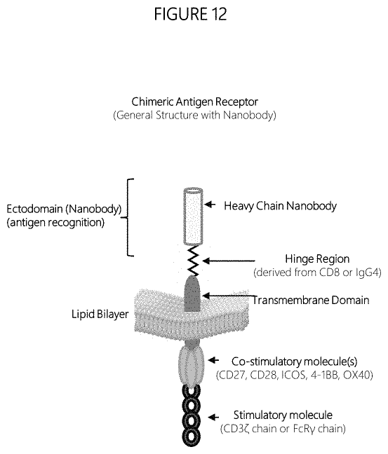

To increase affinity of the VERRs, modifications can be included such as glycosylations. A combination of scFv and VERR can also be used, for example a heavy or light chain of the antibody from the scFV linked to a VP receptor as shown in FIG. 11C. As well, scFv and VERR can be swapped out for a V_{H}H nanobody, V_{NAR}, affilin (or other affinity targeted [bio]chemical ligands), or CER, or any variable heavy chain region as shown in FIG. 12.

Therefore, the present invention provides for a method of treating an individual with cancer, by administering Hypo-BioNVs including CAR receptors having a VERR with viral receptors of an oncolytic virus to an individual, targeting cancer cells (or endothelial cells of the neovasculature, or cancer cells in the stroma), and treating the cancer.

The present invention provides for a method of targeting cells in an individual, by administering the Hypo-BioNVs to an individual, and targeting cells to be destroyed or treated. The CAR receptor (that may consist of either an scFV, VERR, V_{H}H nanobody, V_{NAR}, affilin (or other affinity targeted [bio]chemical ligands), or CER, or other variable heavy chain region) or TCR ligand can recognize its specific biomarker on the cell to be destroyed or treated. Once the CAR docks/interacts with the biomarker on the cell (the target), it releases its payload (drug, cytokine, peptide, gene editor/gRNA, plasmid etc.) The Hypo-BioNVs can enter the tumor microenvironment without being deactivated and can deliver their payloads with more efficiency than other methods.

The Hypo-BioNVs encapsulate the key potent components of activated T-cells. Unlike CAR therapies that have limited efficacy in the tumor micro-environment, the Hypo-BioNVs of the present invention overcome this issue by packing a lymphocyte punch to diseased cells without side effects associated with current approaches. The Hypo-BioNVs eliminate cytokine storm potential, do not lead to teratoma formation, they provide stable and tailored targeted access to any tumor micro-environment, and they have a higher efficacy of tumor penetration than other delivery systems. The Hypo-BioNVs have the advantages of high frequency and tailored targeting, they are highly adaptable, they are off the shelf allogeneic, they have hypo-immunity, they allow for high quality manufacturing and scalability, and uniform and targeted biodistribution.

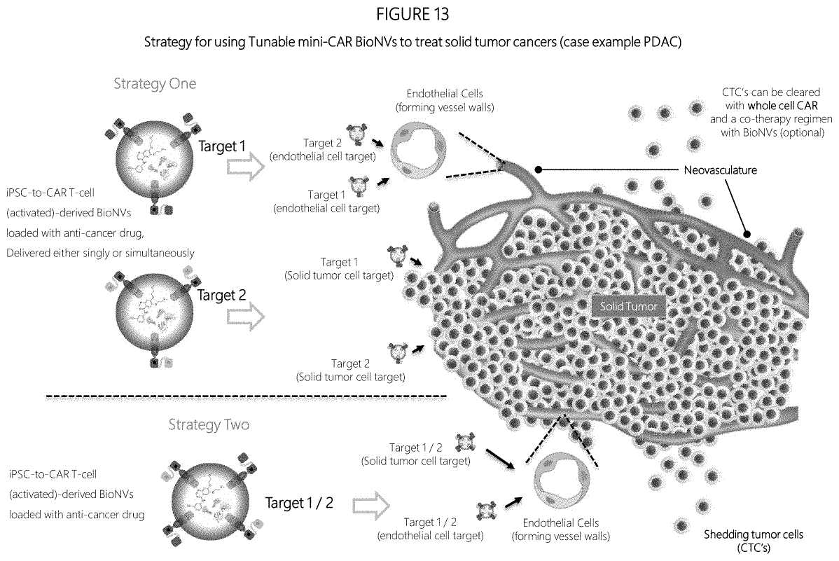

Tunable CAR-loaded Hypo-BioNVs can be used to target and treat cancers. Described above are the strategies for tumor targeting via a CAR (and others such as a TCR) receptor and the loading of therapeutics into Hypo-BioNVs (activated lymphocyte cytokine/chemokine encapsulation, small molecule drug loading, gene editing therapeutics, or any combination of these). One treatment strategy is to use the tunable CAR-loaded Hypo-BioNVs encapsulating anti-cancer drugs (or gene editing, or biologic therapeutics) to target biomarkers (such as TEM8 and/or EIIIB, but not limited to these biomarkers) within the tumor environment and the environment surrounding the tumor as in FIG. 13.

FIG. 13 shows two different strategies for using tunable CAR Hypo-BioNVs in treating solid tumor cancers. In the first strategy, two types of Hypo-BioNVs (administered individually or simultaneously), each derived from iPSC-differentiated CAR T-cells that have been activated with antigen prior to Hypo-BioNV formulation, may be delivered, each targeting two different biomarkers (in this case TEM8 and EIIIB) that each have ability to recognize and treat cancerous endothelial cells of the neovasculature as well as cancer cells of the solid tumor. The Hypo-BioNVs may also recognize cancer cells in the stroma or cells that have shed from the solid tumor. In the second strategy, two or more biomarkers are expressed on the Hypo-BioNVs. In this scenario, the Hypo-BioNVs are also derived from iPSC-differentiated CAR T-cells that have been activated with antigen prior to Hypo-BioNV formulation and contain both TEM8 and EIIIB (but may also contain more or alternate CAR biomarkers). These Hypo-BioNVs recognize and treat cancerous endothelial cells of the neovasculature as well as cancer cells of the solid tumor. The Hypo-BioNVs may also recognize cancer cells in the stroma or cells that have shed from the solid tumor. Further, in both strategies, the Hypo-BioNVs may be loaded with anti-cancer drugs (or gene editing or biologic therapeutics) to increase their efficacy or co-administered with whole cell CAR-lymphocyte therapies, the latter of which could treat/mop-up any cells that are shed from the tumor into circulation. The main advantage of these strategies is to attack the tumor and tumor environment with multiple biomarkers, delivering multiple therapeutics to minimize or prevent the probability of resistance to treatment.

Cancer biomarkers such as EIIIB and/or TEM8 may be effective in simultaneously treating all of 1) endothelial cells of the neovascular mesh that surrounds a solid tumor (thereby starves the tumor of nutrients), 2) tumor cancer cells, 3) cancer cells dispersed in the stroma, 4) cancer cells that shed from the solid tumor (cancer stem cells or circulating tumor cells). Another strategy is to treat each of these environments with a combination of biomarkers (to increase the likelihood of higher efficacies) either: 1) on a single Hypo-BioNV that contains CARs targeting both EIIB and TEM8 (for example but not limited to these). A Hypo-BioNV can contain a single CAR-directed biomarker or multiple CAR-directed biomarkers or, 2) two separate Hypo-BioNVs, where one contains EIIIB and the other contains TEM8 (for example but not limited to these biomarkers), each targeting all the described environments at the same time (broadening the likelihood of targeting success (also, each Hypo-BioNV could be carrying a different anti-cancer drug).

The present invention also provides for a method of treating a central nervous system disease or disorder, by administering the hypo-BioNVs to an individual having a CNS disease or disorder, targeting CNS cells, and treating the CNS disease or disorder.

The hypo-BioNVs can be designed to cross biological barriers and target the central nervous system (CNS) or particular cell types. For example, Alvarez-Ervitti, et al. (2011) describe that exosomes can be decorated with rabies viral glycoprotein (RVG) peptide. Therefore, hypo-BioNVs bearing RVG can be designed to home specifically to the brain, especially to neurons, oligodendrocytes, and microglia, with little nonspecific accumulation in other tissues. An RVG recognition ligand can be added into the CAR construct to provide a bispecific CAR or an additional standalone CAR that is also contained on the surface of the BioNV along with other CARs. Other proteins can be used to create different targets.

Such hypo-BioNVs can be used to carry therapeutics in treating CNS diseases or disorders such as, but not limited to, abulia, achromatopsia, agraphia, akinetopsia, alcoholism, alien hand syndrome, Allan-Herndon-Dudley syndrome, alternating hemiplegia of childhood, Alzheimer's disease, amaurosis fugax, amnesia, amyotrophic lateral sclerosis, aneurysm, Angelman syndrome, anosognosia, aphasia, aphantasia, apraxia, arachnoiditis, Arnold-Chiari malformation, Asomatognosia, Asperger syndrome, ataxia, ATR-16 syndrome, attention deficit hyperactivity disorder, auditory processing disorder, autism spectrum disorder, Behget's disease, Bell's palsy, bipolar disorder, blindsight, brachial plexus injury, brain injury, brain tumor, Brody myopathy, Canavan disease, Capgras delusion, carpal tunnel syndrome, causalgia, central pain syndrome, central pontine myelinolysis, centronuclear myopathy, cephalic disorder, cerebral aneurysm, cerebral arteriosclerosis, cerebral atrophy, cerebral autosomal dominant arteriopathy with subcortical infarcts and leukoencephalopathy, cerebral dysgenesis-neuropathy-ichthyosis-keratoderma syndrome, cerebral gigantism, cerebral palsy, cerebral vasculitis, cerebrospinal fluid leak, cervical spinal stenosis, Charcot-Marie-Tooth disease, Chiari malformation, chorea, chronic fatigue syndrome, chronic inflammatory demyelinating polyneuropathy, chronic pain, cluster headache, Cockayne syndrome, Coffin-Lowry syndrome, coma, complex regional pain syndrome, compression neuropathy, congenital distal spinal muscular atrophy, congenital facial diplegia, corticobasal degeneration, cranial arteritis, craniosynostosis, Creutzfeldt-Jakob disease, cumulative trauma disorders, Cushing's syndrome, cyclic vomiting syndrome, cyclothymic disorder, cytomegalic inclusion body disease, cytomegalovirus infection, Dandy-Walker syndrome, Dawson disease, De Morsier's syndrome, Dejerine-Klumpke palsy, Dejerine-Sottas disease, delayed sleep phase disorder or syndrome, dementia, depression, dermatomyositis, developmental coordination disorder, diabetic neuropathy, diffuse sclerosis, diplopia, disorders of consciousness, distal hereditary motor neuropathy type V, distal spinal muscular atrophy type 1, distal spinal muscular atrophy type 2, Down syndrome, Dravet syndrome, Duchenne muscular dystrophy, dysarthria, dysautonomia, dyscalculia, dysgraphia, dyskinesia, dyslexia, dystonia, empty sella syndrome, encephalitis, encephalocele, encephalopathy, encephalotrigeminal angiomatosis, encopresis, enuresis, epilepsy, epilepsy-intellectual disability in females, Erb's palsy, erythromelalgia, essential tremor, exploding head syndrome, Fabry's disease, Fahr's syndrome, fainting, familial spastic paralysis, fetal alcohol syndrome, febrile seizures, Fisher syndrome, fibromyalgia, Foville's syndrome, fragile X syndrome, fragile X-associated tremor/ataxia syndrome, Friedreich's ataxia, frontotemporal dementia, functional neurological symptom disorder, Gaucher's disease, generalized anxiety disorder, generalized epilepsy with febrile seizures plus, Gerstmann's syndrome, giant cell arteritis, giant cell inclusion disease, globoid cell leukodystrophy, gray matter heterotopia, Guillain-Barré syndrome, head injury, headache, Hemicrania Continua, hemifacial spasm, hemispatial neglect, hereditary motor neuropathies, hereditary spastic paraplegia, heredopathia atactica polyneuritiformis, herpes zoster, herpes zoster oticus, Hirayama syndrome, Hirschsprung's disease, Holmes-Adie syndrome, holoprosencephaly, HTLV-1 associated myelopathy, Huntington's disease, hydranencephaly, hydrocephalus, hypercortisolism, hypoalgesia, hypoesthesia, hypoxia, immune-mediated encephalomyelitis, inclusion body myositis, incontinentia pigmenti, Refsum disease, infantile spasms, inflammatory myopathy, intracranial cyst, intracranial hypertension, isodicentric 15, Joubert syndrome, Karak syndrome, Kearns-Sayre syndrome, Kinsbourne syndrome, Kleine-Levin syndrome, Klippel Feil syndrome, Krabbe disease, Kufor-Rakeb syndrome, Kugelberg-Welander disease, Lafora disease, Lambert-Eaton myasthenic syndrome, Landau-Kleffner syndrome, lateral medullary (Wallenberg) syndrome, learning disabilities, Leigh's disease, Lennox-Gastaut syndrome, Lesch-Nyhan syndrome, leukodystrophy, Leukoencephalopathy with vanishing white matter, Lewy body dementia, lissencephaly, locked-in syndrome, lumbar disc disease, lumbar spinal stenosis, lupus erythematosus-neurological sequelae, Lyme disease, Machado-Joseph disease, macrencephaly, macropsia, Mal de debarquement, megalencephalic leukoencephalopathy with subcortical cysts, megalencephaly, Melkersson-Rosenthal syndrome, Menieres disease, meningitis, Menkes disease, metachromatic leukodystrophy, microcephaly, micropsia, migraine, Miller Fisher syndrome, Mini-stroke (transient ischemic attack), misophonia, mitochondrial myopathy, Mobius syndrome, monomelic amyotrophy, Morvan syndrome, motor skills disorder, Moyamoya disease, mucopolysaccharidoses, multifocal motor neuropathy, multi-infarct dementia, multiple sclerosis, multiple system atrophy, muscular dystrophy, myalgic encephalomyelitis, myasthenia gravis, myelinoclastic diffuse sclerosis, myoclonic encephalopathy of infants, myoclonus, myopathy, myotonia congenita, myotubular myopathy, narcolepsy, Neuro-Behget's disease, neurofibromatosis, neuroleptic malignant syndrome, neuromyotonia, neuronal ceroid lipofuscinosis, neuronal migration disorders, neuropathy, neurosis, Niemann-Pick disease, non-24-hour sleep-wake disorder, nonverbal learning disorder, occipital neuralgia, occult spinal dysraphism sequence, Ohtahara syndrome, olivopontocerebellar atrophy, opsoclonus myoclonus syndrome, optic neuritis, orthostatic hypotension, O'Sullivan-McLeod syndrome, otosclerosis, overuse syndrome, palinopsia, PANDAS, pantothenate kinase-associated neurodegeneration, paramyotonia congenita, paresthesia, Parkinson's disease, paraneoplastic diseases, paroxysmal attacks, Parry-Romberg syndrome, Pelizaeus-Merzbacher disease, periodic paralyses, peripheral neuropathy, pervasive developmental disorders, phantom limb/phantom pain, photic sneeze reflex, phytanic acid storage disease, Pick's disease, pinched nerve, pituitary tumors, polyneuropathy, PMG, polio, polymicrogyria, polymyositis, porencephaly, post-polio syndrome, postherpetic neuralgia, posttraumatic stress disorder, postural hypotension, postural orthostatic tachycardia syndrome, Prader-Willi syndrome, primary lateral sclerosis, prion diseases, progressive hemifacial atrophy, progressive multifocal leukoencephalopathy, progressive supranuclear palsy, prosopagnosia, pseudotumor cerebri, quadrantanopia, quadriplegia, rabies, radiculopathy, Ramsay Hunt syndrome type I, Ramsay Hunt syndrome type II, Rasmussen encephalitis, reflex neurovascular dystrophy, Refsum disease, REM sleep behavior disorder, repetitive stress injury, restless legs syndrome, retrovirus-associated myelopathy, Rett syndrome, Reye's syndrome, rhythmic movement disorder, Romberg syndrome, Saint Vitus dance, Sandhoff disease, Sanfilippo syndrome, Schilder's disease, schizencephaly, sensory processing disorder, septo-optic dysplasia, shaken baby syndrome, shingles, Shy-Drager syndrome, Sjogren's syndrome, sleep apnea, sleeping sickness, snatiation, Sotos syndrome, spasticity, spina bifida, spinal and bulbar muscular atrophy, spinal cord injury, spinal cord tumors, spinal muscular atrophy, spinocerebellar ataxia, split-brain, stiff-person syndrome, stroke, Sturge-Weber syndrome, stuttering, subacute sclerosing panencephalitis, subcortical arteriosclerotic encephalopathy, superficial siderosis, Sydenham's chorea, syncope, synesthesia, syringomyelia, Tardive dyskinesia, Tarlov cyst, tarsal tunnel syndrome, Tay-Sachs disease, temporal arteritis, temporal lobe epilepsy, tetanus, tethered spinal cord syndrome, thalamocortical dysrhythmia, Thomsen disease, thoracic outlet syndrome, Tic Douloureux, tinnitus, Todd's paralysis, Tourette syndrome, toxic encephalopathy, transient ischemic attack, transmissible spongiform encephalopathies, transverse myelitis, traumatic brain injury, tremor, trichotillomania, trigeminal neuralgia, tropical spastic paraparesis, trypanosomiasis, tuberous sclerosis, Unverricht-Lundborg disease, vestibular schwannoma, Viliuisk encephalomyelitis, visual snow, Von Hippel-Lindau disease, Wallenberg's syndrome, Wernicke's encephalopathy, West syndrome, whiplash, Williams syndrome, Wilson's disease, Y-Linked hearing impairment, or Zellweger syndrome.

The hypo-BioNVs can be particularly useful in delivering psychedelics such as, but not limited to, lysergic acid diethylamide (LSD), psilocybin, psilocin, mescaline, 5-methoxy-N,N-dimethyltryptamine (5-MeO-DMT), dimethyltryptamine (DMT), 2,5-dimethoxy-4-iodoamphetamine (DOI), 2,5-dimethoxy-4-bromoamphetamie (DOB), ibogaine, ketamine, salts thereof, tartrates thereof, solvates thereof, isomers thereof, analogs thereof, homologues thereof, or deuterated forms thereof. The psychedelics can be used in treating many CNS disorders listed above as well as addiction disorders and post-traumatic stress disorder.

The gene editors that can be used in engineering the iPSCs are as follows. Once the iPSCs are constructed, gene editor expression cassettes (may or may not be drug regulated) can also be incorporated stably. The iPSC line will now have a gene editor expression cassette that can be turned on. Once turned on, the editor (and gRNAs) will be over-expressed in the cell. The cell is then treated to produce Hypo-BioNVs and the Hypo-BioNVs now have the gene editor with the desired gRNA packaged in them, for delivery as a therapeutic to its intended cell target. Any gene editor listed below will work in this capacity.

Zinc finger nuclease (ZFN) creates double-strand breaks at specific DNA locations. A ZFN has two functional domains, a DNA-binding domain that recognizes a 6 bp DNA sequence, and a DNA-cleaving domain of the nuclease Fok I.

TALENs (transcription activator-like effector nucleases) include a TAL effector DNA-binding domain fused to a DNA cleavage domain that create double strand breaks in DNA.

Human WRN is a RecQ helicase encoded by the Werner syndrome gene. It is implicated in genome maintenance, including replication, recombination, excision repair and DNA damage response. These genetic processes and expression of WRN are concomitantly upregulated in many types of cancers. Therefore, it has been proposed that targeted destruction of this helicase could be useful for elimination of cancer cells. Reports have applied the external guide sequence (EGS) approach in directing an RNase P RNA to efficiently cleave the WRN mRNA in cultured human cell lines, thus abolishing translation and activity of this distinctive 3′-5′ DNA helicase-nuclease.

The Class 2 type VI-A CRISPR/Cas effector “C2c2” demonstrates an RNA-guided RNase function and can be packaged and delivered as a therapeutic in the iPSCs through cassettes as described above. C2c2 from the bacterium Leptotrichia shahii provides interference against RNA phage. In vitro biochemical analysis show that C2c2 is guided by a single crRNA and can be programmed to cleave ssRNA targets carrying complementary protospacers. In bacteria, C2c2 can be programmed to knock down specific mRNAs. Cleavage is mediated by catalytic residues in the two conserved HEPN domains, mutations in which generate catalytically inactive RNA-binding proteins. The RNA-focused action of C2c2 complements the CRISPR-Cas9 system, which targets DNA, the genomic blueprint for cellular identity and function. The ability to target only RNA, which helps carry out the genomic instructions, offers the ability to specifically manipulate RNA in a high-throughput manner—and manipulate gene function more broadly. These results demonstrate the capability of C2c2 as a new RNA-targeting tools.

Another Class 2 type V-B CRISPR/Cas effector “C2c1” can also be used in the present invention for editing DNA. C2c1 contains RuvC-like endonuclease domains related distantly to Cpf1 (described below). C2c1 can target and cleave both strands of target DNA site-specifically. According to Yang et al 2016 (36), a crystal structure confirms Alicyclobacillus acidoterrestris C2c1 (AacC2c1) binds to sgRNA as a binary complex and targets DNAs as ternary complexes, thereby capturing catalytically competent conformations of AacC2c1 with both target and non-target DNA strands independently positioned within a single RuvC catalytic pocket. Yang et al 2016 (36) confirms that C2c1-mediated cleavage results in a staggered seven-nucleotide break of target DNA, crRNA adopts a pre-ordered five-nucleotide A-form seed sequence in the binary complex, with release of an inserted tryptophan, facilitating zippering up of 20-bp guide RNA:target DNA heteroduplex on ternary complex formation, and that the PAM-interacting cleft adopts a “locked” conformation on ternary complex formation.

C2c3 is a gene editor effector of type V-C that is distantly related to C2c1 and contains RuvC-like nuclease domains. C2c3 is also similar to the CasY.1-CasY.6 group described below.

“CRISPR Cas9” as used herein refers to Clustered Regularly Interspaced Short Palindromic Repeat (CRISPR)-associated endonuclease Cas9. In bacteria the CRISPR/Cas loci encode RNA-guided adaptive immune systems against mobile genetic elements (viruses, transposable elements and conjugative plasmids). Three types (I-III) of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA). The CRISPR-associated endonuclease, Cas9, belongs to the type II CRISPR/Cas system and has strong endonuclease activity to cut target DNA. Cas9 is guided by a mature crRNA that contains about 20 base pairs (bp) of unique target sequence (called spacer) and a trans-activated small RNA (tracrRNA) that serves as a guide for ribonuclease III-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd nucleotide from PAM). The crRNA and tracrRNA can be expressed separately or engineered into an artificial fusion small guide RNA (sgRNA) via a synthetic stem loop (AGAAAU) to mimic the natural crRNA/tracrRNA duplex. Such sgRNA, like shRNA, can be synthesized or in vitro transcribed for direct RNA transfection or expressed from U6 or H1-promoted RNA expression vector, although cleavage efficiencies of the artificial sgRNA are lower than those for systems with the crRNA and tracrRNA expressed separately.

CRISPR/Cpf1 is a DNA-editing technology analogous to the CRISPR/Cas9 system, characterized in 2015 by Feng Zhang's group from the Broad Institute and MIT. Cpf1 is an RNA-guided endonuclease of a class II CRISPR/Cas system. This acquired immune mechanism is found in Prevotella and Francisella bacteria. It prevents genetic damage from viruses. Cpf1 genes are associated with the CRISPR locus, coding for an endonuclease that use a guide RNA to find and cleave viral DNA. Cpf1 is a smaller and simpler endonuclease than Cas9, overcoming some of the CRISPR/Cas9 system limitations. CRISPR/Cpf1 could have multiple applications, including treatment of genetic illnesses and degenerative conditions.

A CRISPR/TevCas9 system can also be used. In some cases, it has been shown that once CRISPR/Cas9 cuts DNA in one spot, DNA repair systems in the cells of an organism will repair the site of the cut. The TevCas9 enzyme was developed to cut DNA at two sites of the target so that it is harder for the cells' DNA repair systems to repair the cuts (34). The TevCas9 nuclease is a fusion of a I-Tevi nuclease domain to Cas9.

The Cas9 nuclease can have a nucleotide sequence identical to the wild type Streptococcus pyrogenes sequence. In some embodiments, the CRISPR-associated endonuclease can be a sequence from other species, for example other Streptococcus species, such as thermophilus; Psuedomona aeruginosa, Escherichia coli, or other sequenced bacteria genomes and archaea, or other prokaryotic microorganisms. Alternatively, the wild type Streptococcus pyrogenes Cas9 sequence can be modified. The nucleic acid sequence can be codon optimized for efficient expression in mammalian cells, i.e., “humanized.” A humanized Cas9 nuclease sequence can be for example, the Cas9 nuclease sequence encoded by any of the expression vectors listed in Genbank accession numbers KM099231.1 GI:669193757; KM099232.1 GI:669193761; or KM099233.1 GI:669193765. Alternatively, the Cas9 nuclease sequence can be for example, the sequence contained within a commercially available vector such as PX330 or PX260 from Addgene (Cambridge, Mass.). In some embodiments, the Cas9 endonuclease can have an amino acid sequence that is a variant or a fragment of any of the Cas9 endonuclease sequences of Genbank accession numbers KM099231.1 GI:669193757; KM099232.1 GI:669193761; or KM099233.1 GI:669193765 or Cas9 amino acid sequence of PX330 or PX260 (Addgene, Cambridge, Mass.). The Cas9 nucleotide sequence can be modified to encode biologically active variants of Cas9, and these variants can have or can include, for example, an amino acid sequence that differs from a wild type Cas9 by virtue of containing one or more mutations (e.g., an addition, deletion, or substitution mutation or a combination of such mutations). One or more of the substitution mutations can be a substitution (e.g., a conservative amino acid substitution). For example, a biologically active variant of a Cas9 polypeptide can have an amino acid sequence with at least or about 50% sequence identity (e.g., at least or about 50%, 55%, 60%, 65%, 70%, 75%, 80%, 85%, 90%, 95%, 97%, 98%, or 99% sequence identity) to a wild type Cas9 polypeptide. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. The amino acid residues in the Cas9 amino acid sequence can be non-naturally occurring amino acid residues. Naturally occurring amino acid residues include those naturally encoded by the genetic code as well as non-standard amino acids (e.g., amino acids having the D-configuration instead of the L-configuration). The present peptides can also include amino acid residues that are modified versions of standard residues (e.g., pyrrolysine can be used in place of lysine and selenocysteine can be used in place of cysteine). Non-naturally occurring amino acid residues are those that have not been found in nature, but that conform to the basic formula of an amino acid and can be incorporated into a peptide. These include D-alloisoleucine (2R,3S)-2-amino-3-methylpentanoic acid and L-cyclopentyl glycine (S)-2-amino-2-cyclopentyl acetic acid. For other examples, one can consult textbooks or the worldwide web (a site is currently maintained by the California Institute of Technology and displays structures of non-natural amino acids that have been successfully incorporated into functional proteins). The Cas-9 can also be any shown in TABLE 5 below.

TABLE 5

| Variant No. | Four Alanine Substitution Mutants | Tested* |
|---|---|---|
| | (compared to WT Cas9) | |
| 1 | SpCas9 N497A, R661A, Q695A, | YES |
| | Q926A | |
| 2 | SpCas9 N497A, R661A, Q695A, | YES |
| | Q926A + D1135E | |
| 3 | SpCas9 N497A, R661A, Q695A, | YES |
| | Q926A + L169A | |
| 4 | SpCas9 N497A, R661A, Q695A, | YES |
| | Q926A + Y450A | |
| 5 | SpCas9 N497A, R661A, Q695A, | Predicted |
| | Q926A + M495A | |
| 6 | SpCas9 N497A, R661A, Q695A, | Predicted |
| | Q926A + M694A | |
| 7 | SpCas9 N497A, R661A, Q695A, | Predicted |
| | Q926A + H698A | |
| 8 | SpCas9 N497A, R661A, Q695A, | Predicted |
| | Q926A + D1135E + L169A | |
| 9 | SpCas9 N497A, R661A, Q695A, | Predicted |
| | Q926A + D1135E + Y450A | |
| 10 | SpCas9 N497A, R661A, Q695A, | Predicted |
| | Q926A + D1135E + M495A | |
| 11 | SpCas9 N497A, R661A, Q695A, | Predicted |
| | Q926A + D1135E + M694A | |
| 12 | SpCas9 N497A, R661A, Q695A, | Predicted |
| | Q926A + D1135E + M698A | |
| | Three Alanine Substitution Mutants | Tested* |
| | (compared to WT Cas9) | |
| 13 | SpCas9 R661A, Q695A, Q926A | No (on |
| | | target only) |
| 14 | SpCas9 R661A, Q695A, Q926A + | Predicted |
| | D1135E | |
| 15 | SpCas9 R661A, Q695A, Q926A + | Predicted |
| | L169A | |
| 16 | SpCas9 R661A, Q695A, Q926A + | Predicted |
| | Y450A | |
| 17 | SpCas9 R661A, Q695A, Q926A + | Predicted |
| | M495A | |
| 18 | SpCas9 R661A, Q695A, Q926A + | Predicted |
| | M694A | |
| 19 | SpCas9 R661A, Q695A, Q926A + | Predicted |
| | H698A | |
| 20 | SpCas9 R661A, Q695A, Q926A + | Predicted |
| | D1135E + L169A | |
| 21 | SpCas9 R661A, Q695A, Q926A + | Predicted |
| | D1135E + Y450A | |
| 22 | SpCas9 R661A, Q695A, Q926A + | Predicted |
| | D1135E + M495A | |
| 23 | SpCas9 R661A, Q695A, Q926A + | Predicted |
| | D1135E + M694A | |

Although the RNA-guided endonuclease Cas9 has emerged as a versatile genome-editing platform, some have reported that the size of the commonly used Cas9 from Streptococcus pyogenes (SpCas9) limits its utility for basic research and therapeutic applications that use the highly versatile adeno-associated virus (AAV) delivery vehicle. Accordingly, the six smaller Cas9 orthologues have been used and reports have shown that Cas9 from Staphylococcus aureus (SaCas9) can edit the genome with efficiencies similar to those of SpCas9, while being more than 1 kilobase shorter. SaCas9 is 1053 bp, whereas SpCas9 is 1358 bp.

The Cas9 nuclease sequence, or any of the gene editor effector sequences described herein, can be a mutated sequence. For example, the Cas9 nuclease can be mutated in the conserved HNH and RuvC domains, which are involved in strand specific cleavage. For example, an aspartate-to-alanine (D10A) mutation in the RuvC catalytic domain allows the Cas9 nickase mutant (Cas9n) to nick rather than cleave DNA to yield single-stranded breaks, and the subsequent preferential repair through HDR can potentially decrease the frequency of unwanted indel mutations from off-target double-stranded breaks. In general, mutations of the gene editor effector sequence can minimize or prevent off-targeting.

The gene editor effector can also be Archaea Cas9. The size of Archaea Cas9 is 950aa ARMAN 1 and 967aa ARMAN 4. The Archaea Cas9 can be derived from ARMAN-1 (Candidatus Micrarchaeum acidiphilum ARMAN-1) or ARMAN-4 (Candidatus Parvarchaeum acidiphilum ARMAN-4).

The gene editor effector can be a Saliogase, described in WO2021226141 to Higgins, et al. Saliogases can integrate DNA sequences without causing double strand DNA breaks or using host DNA repair pathways of homologous recombination. A lipid nanoparticle or BioNV can include the donor DNA and helper RNA that encodes the Saliogase for delivery to a target cell. Saliogases have the advantage of minimal risk of immune response, increased reliability and efficiency, and the ability to deliver any length of DNA sequence. WO2021226141 discloses an enzyme capable of targeted genomic integration by transposition (e.g., a recombinase, an integrase, or a transposase enzyme), as a monomer or a dimer, that can be fused with a transcription activator-like effector proteins (TALE) DNA binding domain (DBD) or a dCas9/g RNA to thereby create a chimeric enzyme capable of a site- or locus-specific transposition. The enzyme (e.g., a chimeric transposase) utilizes the specificity of TALE DBD to certain sites within a host genome, which allows using DBDs to target any desired location in the genome. In this way, the chimeric transposase allows achieving targeted integration of a transgene. Mutations can be included that confer hyperactivity. Dual transposon/transposase (or “donor/helper”) plasmid systems insert a transgene flanked by inverted terminal ends (“ends”), such as TTAA tetranucleotide sites, without leaving a DNA footprint in the human genome. The transposase enzyme is transiently expressed (on the same or a different vector from a vector encoding the transposon) and it catalyzes the insertion events from the donor plasmid to the host genome. TALE or dCas9/gRNA DBDs can cause the enzyme capable of transposition (e.g., a chimeric transposase) to bind specifically to human genomic safe harbor sites.

Any of the gene editor effectors herein can also be tagged with Tev or any other suitable homing protein domains. According to Wolfs et al 2016 (34), Tev is an RNA-guided dual active site nuclease that generates two noncompatible DNA breaks at a target site, effectively deleting the majority of the target site such that it cannot be regenerated.

The gene editor can also be any gene editor that is derived from or designed in silico either from extrapolating from existing domain and amino acid sequence analysis, or an entirely engineered (unique amino acid composition and domain structure) using artificial intelligence design.

Vectors containing nucleic acids such as those described herein also are provided. A “vector” is a replicon, such as a plasmid, phage, or cosmid, into which another DNA segment may be inserted to bring about the replication of the inserted segment. Generally, a vector is capable of replication when associated with the proper control elements. Suitable vector backbones include, for example, those routinely used in the art such as plasmids, viruses, artificial chromosomes, BACs, YACs, or PACs. The term “vector” includes cloning and expression vectors, as well as viral vectors and integrating vectors. An “expression vector” is a vector that includes a regulatory region. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

The vectors provided herein also can include, for example, origins of replication, scaffold attachment regions (SARs), and/or markers. A marker gene can confer a selectable phenotype on a host cell. For example, a marker can confer biocide resistance, such as resistance to an antibiotic (e.g., kanamycin, G418, bleomycin, or hygromycin). As noted above, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

Additional expression vectors also can include, for example, segments of chromosomal, non-chromosomal and synthetic DNA sequences. Suitable vectors include derivatives of SV40 and known bacterial plasmids, e.g., E. coli plasmids col E1, pCR1, pBR322, pMal-C2, pET, pGEX, pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2p plasm id or derivatives thereof, vectors useful in eukaryotic cells, such as vectors useful in insect or mammalian cells; vectors derived from combinations of plasmids and phage DNAs, such as plasmids that have been modified to employ phage DNA or other expression control sequences.

The vector can also include a regulatory region. The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, nuclear localization signals, and introns.

As used herein, the term “operably linked” refers to positioning of a regulatory region and a sequence to be transcribed in a nucleic acid to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically comprises at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

Vectors include, for example, viral vectors (such as adenoviruses (“Ad”), adeno-associated viruses (AAV), and vesicular stomatitis virus (VSV) and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. As described and illustrated in more detail below, such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al 2003 (2). A large variety of such vectors are known in the art and are generally available.

A “recombinant viral vector” refers to a viral vector comprising one or more heterologous gene products or sequences. Since many viral vectors exhibit size-constraints associated with packaging, the heterologous gene products or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying gene products necessary for replication and/or encapsidation). Modified viral vectors in which a polynucleotide to be delivered is carried on the outside of the viral particle have also been described (0).

Suitable nucleic acid delivery systems include recombinant viral vector, typically sequence from at least one of an adenovirus, adenovirus-associated virus (AAV), helper-dependent adenovirus, retrovirus, or hemagglutinating virus of Japan-liposome (HVJ) complex. In such cases, the viral vector comprises a strong eukaryotic promoter operably linked to the polynucleotide e.g., a cytomegalovirus (CMV) promoter. The recombinant viral vector can include one or more of the polynucleotides therein, preferably about one polynucleotide. In some embodiments, the viral vector used in the invention methods has a pfu (plague forming units) of from about 10⁸ to about 5×10¹⁰ pfu. In embodiments in which the polynucleotide is to be administered with a non-viral vector, use of between from about 0.1 nanograms to about 4000 micrograms will often be useful e.g., about 1 nanogram to about 100 micrograms.

Additional vectors include viral vectors, fusion proteins and chemical conjugates. Retroviral vectors include Moloney murine leukemia viruses and HIV-based viruses. One HIV-based viral vector comprises at least two vectors wherein the gag and pol genes are from an HIV genome and the env gene is from another virus. DNA viral vectors include pox vectors such as orthopox or avipox vectors, herpesvirus vectors such as a herpes simplex I virus (HSV) vector [Geller et al 1995 (13); Lim et al 1995 (21), Glover et al 1995 (16); Geller et al 1993 (14); Geller et al 1990 (15)], Adenovirus Vectors [LaSalle et al 1993 (11); Davidson et al 1993 (0); Yang et al 1995 (37)] and Adeno-associated Virus Vectors [Kaplitt et al 1994 (20)].

Pox viral vectors introduce the gene into the cell's cytoplasm. Avipox virus vectors result in only a short-term expression of the nucleic acid. Adenovirus vectors, adeno-associated virus vectors and herpes simplex virus (HSV) vectors may be an indication for some invention embodiments. The adenovirus vector results in a shorter-term expression (e.g., less than about a month) than adeno-associated virus, in some embodiments, may exhibit much longer expression. The particular vector chosen will depend upon the target cell and the condition being treated. The selection of appropriate promoters can readily be accomplished. An example of a suitable promoter is the 763-base-pair cytomegalovirus (CMV) promoter. Other suitable promoters which may be used for gene expression include, but are not limited to, the Rous sarcoma virus (RSV) (0), the SV40 early promoter region, the herpes thymidine kinase promoter, the regulatory sequences of the metallothionein (MMT) gene, prokaryotic expression vectors such as the β-lactamase promoter, the tac promoter, promoter elements from yeast or other fungi such as the GAL4 promoter, the ADH (alcohol dehydrogenase) promoter, PGK (phosphoglycerol kinase) promoter, alkaline phosphatase promoter; and the animal transcriptional control regions, which exhibit tissue specificity and have been utilized in transgenic animals: elastase I gene control region which is active in pancreatic acinar cells, insulin gene control region which is active in pancreatic beta cells, immunoglobulin gene control region which is active in lymphoid cells, mouse mammary tumor virus control region which is active in testicular, breast, lymphoid and mast cells, albumin gene control region which is active in liver, alpha-fetoprotein gene control region which is active in liver, alpha 1-antitrypsin gene control region which is active in the liver, beta-globin gene control region which is active in myeloid cells, myelin basic protein gene control region which is active in oligodendrocyte cells in the brain, myosin light chain-2 gene control region which is active in skeletal muscle, and gonadotropic releasing hormone gene control region which is active in the hypothalamus. Certain proteins can be expressed using their native promoter. Other elements that can enhance expression can also be included such as an enhancer or a system that results in high levels of expression such as a tat gene and tar element. This cassette can then be inserted into a vector, e.g., a plasmid vector such as, pUC19, pUC118, pBR322, or other known plasmid vectors, that includes, for example, an E. coli origin of replication. See, Sambrook et al 1989 (28). The plasmid vector may also include a selectable marker such as the β-lactamase gene for ampicillin resistance, provided that the marker polypeptide does not adversely affect the metabolism of the organism being treated. The cassette can also be bound to a nucleic acid binding moiety in a synthetic delivery system, such as the system disclosed in WO 95/22618.

If desired, the polynucleotides of the invention can also be used with a microdelivery vehicle such as cationic liposomes and adenoviral vectors. For a review of the procedures for liposome preparation, targeting and delivery of contents, see Mannino et al 1988 (23). See also, Feigner et al 1989 (9) and Maurer et al 1989 (24).

Replication-defective recombinant adenoviral vectors can be produced in accordance with known techniques (26, 27, 32).

Another delivery method is to use single stranded DNA producing vectors which can produce the expressed products intracellularly. See for example, Chen et al 2003 (2), which is incorporated herein, by reference, in its entirety.

As described above, the compositions of the present invention can be prepared in a variety of ways known to one of ordinary skill in the art. Regardless of their original source or the way they are obtained, the compositions of the invention can be formulated in accordance with their use. For example, the nucleic acids and vectors described above can be formulated within compositions for application to cells in tissue culture or for administration to a patient or subject. Any of the pharmaceutical compositions of the invention can be formulated for use in the preparation of a medicament, and particular uses are indicated below in the context of treatment, e.g., the treatment of a subject having a virus or at risk for contracting a virus. When employed as pharmaceuticals, any of the nucleic acids and vectors can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intra-arterial, subcutaneous, intraperitoneal or intramuscular injection or infusion; or intracranial, e.g., intrathecal or intraventricular administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids, powders, and the like. Conventional pharmaceutical carriers, aqueous, powder or oily bases, thickeners and the like may be necessary or desirable.

This invention also includes pharmaceutical compositions which contain, as the active ingredient, nucleic acids and vectors described herein in combination with one or more pharmaceutically acceptable carriers. The terms “pharmaceutically acceptable” (or “pharmacologically acceptable”) refer to molecular entities and compositions that do not produce an adverse, allergic or other untoward reaction when administered to an animal or a human, as appropriate. The methods and compositions disclosed herein can be applied to a wide range of species, e.g., humans, non-human primates (e.g., monkeys), horses or other livestock, dogs, cats, ferrets or other mammals kept as pets, rats, mice, or other laboratory animals. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance. In making the compositions of the invention, the active ingredient is typically mixed with an excipient, diluted by an excipient or enclosed within such a carrier in the form of, for example, a capsule, tablet, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), lotions, creams, ointments, gels, soft and hard gelatin capsules, suppositories, sterile injectable solutions, and sterile packaged powders. As is known in the art, the type of diluent can vary depending upon the intended route of administration. The resulting compositions can include additional agents, such as preservatives. In some embodiments, the carrier can be, or can include a lipid-based or polymer-based colloid. In some embodiments, the carrier material can be a colloid formulated as a liposome, a hydrogel, a microparticle, a nanoparticle, or a block copolymer micelle. As noted, the carrier material can form a capsule, and that material may be a polymer-based colloid.

The Hypo-BioNVs may also be applied to a surface of a device (e.g., a catheter) or contained within a pump, patch, or other drug delivery device. The nucleic acids and vectors of the invention can be administered alone, or in a mixture, in the presence of a pharmaceutically acceptable excipient or carrier (e.g., physiological saline). The excipient or carrier is selected based on the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

### BODY REFERENCES

Bonilla W. V. et al. Heterologous arenavirus vector prime-boost overrules self-tolerance for efficient tumor-specific CD8 T cell attack. Cell Rep Med. 2021 Mar. 3; 2(3):100209.
2. Chen Y. et al. Expression of ssDNA in mammalian cells. BioTechniques. 2003 January; 34(1):167-71.
3. Chmielewski M. et al. TRUCKs: the fourth generation of CARs. Expert Opin Biol Ther. 2015; 15(8):1145-54.
4. Corbo C. et al. Engineered biomimetic nanovesicles show intrinsic anti-inflammatory properties for the treatment of inflammatory bowel diseases. Nanoscale. 2017 Oct. 5; 9(38):14581-14591.
5. Curiel D. et al. Adenovirus enhancement of transferrin-polylysine-mediated gene delivery. Proc Natl Acad Sci USA. 1991 Oct. 1; 88(19):8850-4.
6. Davidson, et al. A model system for in vivo gene transfer into the central nervous system using an adenoviral vector. Nat. Genet. 1993 March; 3(3):219-23.
7. Davis, et al. Direct gene transfer into skeletal muscle in vivo: factors affecting efficiency of transfer and stability of expression. Hum Gene Ther. 1993 April; 4(2):151-9.
8. Deuse T. et al. Hypoimmunogenic derivatives of induced pluripotent stem cells evade immune rejection in fully immunocompetent allogeneic recipients. Nat Biotechnol. 2019 March; 37(3):252-258.
9. Feigner et al. Bethesda Res. Lab. Focus. 1989 11(2):21
10. Fu W. et al. CAR exosomes derived from effector CAR-T cells have potent antitumour effects and low toxicity. Nat Commun. 2019 Sep. 25; 10(1):4355.
11. G Le Gal La Salle et al. An adenovirus vector for gene transfer into neurons and glia in the brain. Science. 1993 Feb. 12; 259(5097):988-90.
12. Gandhapudi S. et al. Antigen Priming with Enantiospecific Cationic Lipid Nanoparticles Induces Potent Antitumor CTL Responses through Novel Induction of a Type I IFN Response. J Immunol. 2019 Jun. 15; 202(12):3524-3536.
13. Geller A. I. et al. An HSV-1 vector expressing tyrosine hydroxylase causes production and release of L-dopa from cultured rat striatal cells. J. Neurochem. 1995 February; 64(2):487-96.
14. Geller A. I. et al., Long-term increases in neurotransmitter release from neuronal cells expressing a constitutively active adenylate cyclase from a herpes simplex virus type 1 vector. Proc Natl. Acad. Sci.: U.S.A. 1993 Aug. 15; 90(16):7603-7.
15. Geller A. I., et al. Infection of cultured central nervous system neurons with a defective herpes simplex virus 1 vector results in stable expression of Escherichia coli beta-galactosidase. Proc Natl. Acad. Sci USA: 1990 February; 87(3):1149-53.
16. Glover D. et al. Oxford Univ. Press, Oxford England. 1995.
17. Goh W. J. et al. Bioinspired Cell-Derived Nanovesicles versus Exosomes as Drug Delivery Systems: a Cost-Effective Alternative. Sci Rep. 2017 Oct. 30; 7(1):14322.
18. Goh W. J. et al. Doxorubicin-loaded cell-derived nanovesicles: an alternative targeted approach for anti-tumor therapy. Int J Nanomedicine. 2017 Apr. 4; 12:2759-2767.
19. Jang S. C. et al. Bioinspired exosome-mimetic nanovesicles for targeted delivery of chemotherapeutics to malignant tumors. ACS Nano. 2013 Sep. 24; 7(9):7698-710.
20. Kaplitt M. G., et al. Long-term gene expression and phenotypic correction using adeno-associated virus vectors in the mammalian brain. Nat. Genet. 1994 October; 8(2):148-54.
21. Lim F., et al. DNA Cloning: Mammalian Systems. 1995.
22. Lunavat T. R. et al. RNAi delivery by exosome-mimetic nanovesicles—Implications for targeting c-Myc in cancer. Biomaterials. 2016 September; 102:231-8.
23. Mannino and Gould-Fogerite. Liposome mediated gene transfer. BioTechniques. 1988; 6(7):682-90.
24. Maurer R. A. et al. Bethesda Res. Lab. Focus. 1989 11(2):25.
25. Molinaro R. et al. Design and Development of Biomimetic Nanovesicles Using a Microfluidic Approach. Adv Mater. 2018 April; 30(15).
26. Quantin, et al. Adenovirus as an expression vector in muscle cells in vivo. Proc. Natl. Acad. Sci. USA, 1992 Apr. 1; 89(7):2581-4.
27. Rosenfeld, et al. In vivo transfer of the human cystic fibrosis transmembrane conductance regulator gene to the airway epithelium. Cell. 1992 Jan. 10; 68(1):143-55.
28. Sambrook, et al. Molecular Cloning: A Laboratory Manual. Cold Spring Harbor Laboratory Press. 1989.
29. Singh N. et al. Antigen-independent activation enhances the efficacy of 4-1 BB-costimulated CD22 CAR T cells. Nat Med. 2021 May; 27(5):842-850.
30. Sonntag F. et al. AAV type 2 capsids with externalized VP1/VP2 trafficking domains are generated prior to passage through the cytoplasm and are maintained until the uncoating occurs in the nucleus. J Virol. 2006 November; 80(22):11040-54.
31. Stahnke S. et al. Intrinsic phospholipase A2 activity of AAV is involved in endosomal escape of incoming particles. Virology. 2011 Jan. 5; 409(1):77-83.
32. Stratford-Perricadet, et al. Widespread long-term gene transfer to mouse skeletal muscles and heart. J. Clin. Invest. 1992 August; 90(2):626-30. J Clin Invest.
33. Thalhauser S. et al. Presentation of HIV-1 Envelope Trimers on the Surface of Silica Nanoparticles. J Pharm Sci. 2020 January; 109(1):911-921.
34. Wolfs J. et al. Biasing genome-editing events toward precise length deletions with an RNA-guided TevCas9 dual nuclease. Proc Natl Acad Sci USA. 2016 Dec. 27; 113(52):14988-14993.
35. Wu J. Y. et al. Exosome-Mimetic Nanovesicles from Hepatocytes promote hepatocyte proliferation in vitro and liver regeneration in vivo. Sci Rep. 2018 Feb. 6; 8(1):2471.
36. Yang H. et al. PAM-Dependent Target DNA Recognition and Cleavage by C2c1 CRISPR-Cas Endonuclease. Cell. 2016 Dec. 15; 167(7):1814-1828.e12.
37. Yang, et al. Cellular and humoral immune responses to viral antigens create barriers to lung-directed gene therapy with recombinant adenoviruses. J. Virol. 1995 April; 69(4):2004-15.
38. Zadori Z. et al. A viral phospholipase A2 is required for parvovirus infectivity. Dev Cell. 2001 August; 1(2):291-302.
39. Zhang P. et al. Genetically Engineered Liposome-like Nanovesicles as Active Targeted Transport Platform. Adv Mater. 2018 February; 30(7).

### TABLE 4 REFERENCES

A. Mei Z. et al. MUC1 as a target for CAR-T therapy in head and neck squamous cell carcinoma. Cancer Med. 2020 January; 9(2):640-652
B. Wang K. et al. GD2-specific CAR T cells encapsulated in an injectable hydrogel control retinoblastoma and preserve vision. Nat Cancer. 2020 October; 1(10):990-997.

What is claimed is:

1. A method of generating allogenic biomimetic nanovesicles (BioNVs) from gene edited iPSCs including the steps of:
disrupting cell membranes of the gene edited iPSCs using a method chosen from the group consisting of sonicating, adaptive focused acoustics technology, extrusion, serial extrusion, rupturing by detergents or enzymes, electroporation, and combinations thereof; and
purifying the BioNVs by a method chosen from the group consisting of microfiltration, affinity chromatography, size exclusion chromatography, gel purification, centrifugation, and combinations thereof.

2. The method of claim 1, wherein the iPSCs include surface ligands that target cells or tissues.

3. The method of claim 1, wherein the BioNVs include a surface coated with CAR ligands chosen from the group consisting of scFV, VERR, V_{H}H nanobody, V_{NAR}, affilin (or other affinity targeted [bio]chemical ligands), CER, and combinations thereof.

4. The method of claim 1, wherein density of CAR expression on the BioNVs is controlled.

5. The method of claim 1, wherein the BioNVs include TCR ligands.

6. The method of claim 1, wherein the BioNVs include a modification chosen from the group consisting of B2M−/− CIITA−/−, CD47+/+, PD1−/−, Trac−/−, CORE Primary CAR Expression Cassette, increased expression of Perforin and Granzyme B Expression, Fas Ligand +ve, increased expression of NCAM, increased or decreased expression of differential regulation of Interleukins, ADVANCE 2^{nd}/3^{rd} Gen CAR Expression Cassette, and Lymphocyte Activation Modifications N/A.

7. The method of claim 1, wherein the BioNvs include knocked out or regulated interleukins.

8. The method of claim 1, wherein the BioNvs include pro-inflammatory interleukins on their surface.

9. The method of claim 1, wherein the BioNvs include green fluorescent protein (GFP).

10. The method of claim 1, further including the step of loading the BioNVs with a therapeutic chosen from the group consisting of DNA, plasmid DNA, RNA, protein, small molecule, and combinations thereof.

11. The method of claim 1, wherein the therapeutic is a pro-inflammatory interleukin.

12. BioNVs produced by the method of claim 1.

13. A composition comprising a therapeutic agent packaged in a hypo-BioNV, wherein said therapeutic agent is chosen from the group consisting of DNA, plasmid DNA, RNA, protein, small molecule, and combinations thereof.

14. The composition of claim 13, wherein said therapeutic agent is a pro-inflammatory interleukin.

15. The composition of claim 13, wherein said therapeutic agent is a gene editor chosen from the group consisting of TALENs, ZFNs, RNase P RNA, C2c1, C2c2, C2c3, Cas9, Cpf1, TevCas9, Archaea Cas9, CasY.1, CasY.2, CasY.3, CasY.4, CasY.5, CasY.6, CasX, Cas omega, Saliogase, orthologs thereof, and homologs thereof.

16. The composition of claim 13, wherein said therapeutic agent is a psychedelic chosen from the group consisting of lysergic acid diethylamide (LSD), psilocybin, psilocin, mescaline, 5-methoxy-N,N-dimethyltryptamine (5-MeO-DMT), dimethyltryptamine (DMT), 2,5-dimethoxy-4-iodoamphetamine (DOI), 2,5-dimethoxy-4-bromoamphetamie (DOB), ibogaine, ketamine, salts thereof, tartrates thereof, solvates thereof, isomers thereof, analogs thereof, homologues thereof, and deuterated forms thereof.

17. The composition of claim 13, wherein said Hypo-BioNVs include a surface coated with CAR ligands chosen from the group consisting of scFV, VERR, V_{H}H nanobody, V_{NAR}, affilin (or other affinity targeted [bio]chemical ligands), CER, and combinations thereof.

18. The composition of claim 13, wherein density of CAR expression on said Hypo-BioNVs is controlled.

19. The composition of claim 13, wherein said Hypo-BioNVs include TCR ligands.

20. The composition of claim 13, wherein said Hypo-BioNVs include a modification chosen from the group consisting of B2M−/− CIITA−/−, CD47+/+, PD1−/−, Trac−/−, CORE Primary CAR Expression Cassette, increased expression of perforin and granzyme B Expression, Fas Ligand +ve, increased expression of NCAM, increased or decreased expression of differential regulation of Interleukins, ADVANCE 2^{nd}/3^{rd} Gen CAR Expression Cassette, Lymphocyte Activation Modifications N/A, and combinations thereof.

21. The composition of claim 13, wherein said Hypo-BioNvs include knocked out or regulated interleukins.

22. The composition of claim 13, wherein said Hypo-BioNvs include pro-inflammatory interleukins on their surface.

23. The composition of claim 13, wherein said Hypo-BioNVs include bispecific CARs/TCRs that recognize two biomarkers on cells.

24. The composition of claim 13, wherein said Hypo-BioNVs include a fusion peptide embedded in a membrane or a charged surface lipid bilayer for facilitating target cell membrane fusion and subsequent release of the therapeutic agent.

25. The composition of claim 13, wherein said Hypo-BioNVs include rabies viral glycoprotein (RVG) peptide.

26. The composition of claim 13, further including a cell-penetrating peptide.

27. The composition of claim 26, wherein the cell-penetrating peptide is chosen from the group consisting of gp120/gp41 and RAAS from CD2v.

28. A method of treating an individual with cancer, an infectious disease, or hereditary disease, including the steps of:
administering Hypo-BioNVs to an individual;
targeting cells chosen from the group consisting of cancer cells, cells that have been biochemically or genetically corrupted by an infectious pathogen, and cells that have hereditary aberrations or genetic mutations; and
treating the cancer, infectious disease, or hereditary disease.

29. The method of claim 28, wherein the Hypo-BioNVs include a therapeutic chosen from the group consisting of DNA, plasmid DNA, RNA, protein, small molecule, and combinations thereof, and further including the step of releasing the therapeutic at the targeted cells.

30. The method of claim 28, wherein the therapeutic is a pro-inflammatory interleukin.

31. The method of claim 28, wherein the therapeutic is a gene editor chosen from the group consisting of TALENs, ZFNs, RNase P RNA, C2c1, C2c2, C2c3, Cas9, Cpf1, TevCas9, Archaea Cas9, CasY.1, CasY.2, CasY.3, CasY.4, CasY.5, CasY.6, CasX, Cas omega, Saliogase, orthologs thereof, and homologs thereof.

32. The method of claim 28, wherein the cancer cell is chosen from the group consisting of adenoid cystic carcinoma, adrenal gland tumors, amyloidosis, anal cancer, appendix cancer, astrocytoma, ataxia-telangiectasia, attenuated familial adenomatous polyposis, Beckwith-Wiedermann Syndrome, bile duct cancer, Birt-Hogg-Dube Syndrome, bladder cancer, bone cancer, brain stem glioma, brain tumors, breast cancer, carcinoid tumors, Carney complex, central nervous system tumors, cervical cancer, colorectal cancer, Cowden syndrome, craniopharyngioma, desmoplastic infantile ganglioglioma, endocrine tumors, ependymoma, esophageal cancer, Ewing sarcoma, eye cancer, eyelid cancer, fallopian tube cancer, familial adenomatous polyposis, familial malignant melanoma, familial non-VHL clear cell renal cell carcinoma, gallbladder cancer, Gardner Syndrome, gastrointestinal stromal tumor, germ cell tumor, gestational trophoblastic disease, head and neck cancer, diffuse gastric cancer, leiomyomatosis and renal cell cancer, mixed polyposis syndrome, pancreatitis, papillary renal cell carcinoma, HIV and AIDS-related cancer, islet cell tumors, juvenile polyposis syndrome, kidney cancer, lacrimal gland tumor, laryngeal and hypopharyngeal cancer, acute lymphoblastic leukemia, acute lymphocytic leukemia, acute myeloid leukemia, B-cell prolymphocytic leukemia, hairy cell leukemia, chronic lymphocytic leukemia, chronic myeloid leukemia, chronic T-cell lymphocytic leukemia, eosinophilic leukemia, Li-Fraumeni Syndrome, liver cancer, lung cancer, Hodgkin lymphoma, Non-Hodgkin lymphoma, Lynch Syndrome, mastocytosis, medulloblastoma, melanoma, meningioma, mesothelioma, Muir-Torre Syndrome, multiple endocrine neoplasia type 1, multiple endocrine neoplasia type 2, multiple myeloma, myelodysplastic syndromes, MYH-associated polyposis, nasal cavity and paranasal sinus cancer, nasopharyngeal cancer, neuroblastoma, neuroendocrine tumors, neurofibromatosis type 1, neurofibromatosis type 2, nevoid basal cell carcinoma syndrome, oral and oropharyngeal cancer, osteosarcoma, ovarian cancer, pancreatic cancer, parathyroid cancer, penile cancer, Peutz-Jeghers Syndrome, pituitary gland tumors, pleuropulmonary blastoma, prostate cancer, retinoblastoma, rhabdomyosarcoma, salivary gland cancer, sarcoma, alveolar soft part and cardiac sarcoma, Kaposi sarcoma, skin cancer, small bowel cancer, stomach cancer, testicular cancer, thymoma, thyroid cancer, tuberous sclerosis syndrome, Turcot Syndrome, unknown primary, uterine cancer, vaginal cancer, Von Hippel-Lindau Syndrome, Wilms tumors, and Xeroderma pigmentosum.

33. The method of claim 28, wherein the infectious pathogen is chosen from the group consisting of influenza, measles, COVID-19, AIDS, amebiasis, anaplasmosis, anthrax, antibiotic resistance, avian influenza, babesiosis, botulism, brucellosis, campylobacter, cat scratch disease, chickenpox, chikungunya, Chlamydia trachomatis, cholera, Clostridium perfringens, conjunctivitis, crusted scabies, cryptosporidiosis, cyclospora, dengue fever, diphtheria, ebola virus disease, E. coli, eastern equine encephalitis (EEE), enterovirus 68, fifth disease, genital herpes, genital warts, giardia, gonorrhea, group A Streptococcus, Guillain-Barré syndrome, Hand, Foot & Mouth Disease, Hansen's disease, hantavirus, lice, hepatitis A, hepatitis B, hepatitis C, herpes, herpes B virus, Hib disease, histoplasmosis, HIV, HPV (Human Papillomavirus), impetigo, Kawasaki syndrome, legionellosis, leprosy, leptospirosis, listeriosis, lyme disease, lymphocytic choriomeningitis (LCMV), malaria, Marburg virus, meningitis, meningococcal disease, MERS (Middle East Respiratory Illness), monkeypox, mononucleosis, MRSA, mumps, Mycoplasma pneumoniae, neisseria meningitis, norovirus, Orf Virus (Sore Mouth), pelvic inflammatory disease (PID), PEP, pertussis, pink eye, plague, pneumococcal disease, powassan virus, psittacosis, Q fever, rabies, raccoon roundworm, rat bite fever, Reye's Syndrome, Rickettsialpox, ringworm, rubella, salmonella, scabies, scarlet fever, shigella, shingles, smallpox, strep throat, syphilis, tetanus, toxoplasmosis, trichinosis, trichomoniasis, tuberculosis, tularemia, varicella, vibriosis, viral hemorrhagic fevers (VHF), West Nile virus, whooping cough, yellow fever, yersiniosis, or zika virus.

34. The method of claim 28, wherein the hereditary disease is chosen from the group consisting of 1p36 deletion syndrome, 18p deletion syndrome, 21-hydroxylase deficiency, Alpha 1-antitrypsin deficiency, AAA syndrome (achalasia-addisonianism-alacrima syndrome), Aarskog-Scott syndrome, ABCD syndrome, Aceruloplasminemia, Acheiropodia, Achondrogenesis type II, achondroplasia, Acute intermittent porphyria, adenylosuccinate lyase deficiency, Adrenoleukodystrophy, Alagille syndrome, ADULT syndrome, Aicardi-Goutières syndrome, Albinism, Alexander disease, alkaptonuria, Alport syndrome, Alternating hemiplegia of childhood, Amyotrophic lateral sclerosis-Frontotemporal dementia, Alström syndrome, Alzheimer's disease, Amelogenesis imperfecta, Aminolevulinic acid dehydratase deficiency porphyria, Androgen insensitivity syndrome, Angelman syndrome, Apert syndrome, Arthrogryposis-renal dysfunction-cholestasis syndrome, Ataxia telangiectasia, Axenfeld syndrome, Beare-Stevenson cutis gyrata syndrome, Beckwith-Wiedemann syndrome, Benjamin syndrome, biotinidase deficiency, Bjornstad syndrome, Bloom syndrome, Birt-Hogg-Dube syndrome, Brody myopathy, Brunner syndrome, CADASIL syndrome, CRASIL syndrome, Chronic granulomatous disorder, Campomelic dysplasia, Canavan disease, Carpenter Syndrome, Cerebral dysgenesis-neuropathy-ichthyosis-keratoderma syndrome (SEDNIK), Cystic fibrosis, Charcot-Marie-Tooth disease, CHARGE syndrome, Chediak-Higashi syndrome, Cleidocranial dysostosis, Cockayne syndrome, Coffin-Lowry syndrome, Cohen syndrome, collagenopathy, types II and XI, Congenital insensitivity to pain with anhidrosis (CIPA), Congenital Muscular Dystrophy, Cornelia de Lange syndrome (CDLS), Cowden syndrome, CPO deficiency (coproporphyria), Cranio-lenticulo-sutural dysplasia, Cri du chat, Crohn's disease, Crouzon syndrome, Crouzonodermoskeletal syndrome (Crouzon syndrome with acanthosis nigricans), Darier's disease, Dent's disease (Genetic hypercalciuria), Denys-Drash syndrome, De Grouchy syndrome, Down Syndrome, Di George's syndrome, Distal hereditary motor neuropathies, Distal muscular dystrophy, Duchenne muscular dystrophy, Dravet syndrome, Edwards Syndrome, Ehlers-Danlos syndrome, Emery-Dreifuss syndrome, Epidermolysis bullosa, Erythropoietic protoporphyria, Fanconi anemia (FA), Fabry disease, Factor V Leiden thrombophilia, Fatal familial insomnia, Familial adenomatous polyposis, Familial dysautonomia, Familial Creutzfeld-Jakob Disease, Feingold syndrome, FG syndrome, Fragile X syndrome, Friedreich's ataxia, G6PD deficiency, Galactosemia, Gaucher disease, Gerstmann-Sträussler-Scheinker syndrome, Gillespie syndrome, Glutaric aciduria, type I and type 2, GRACILE syndrome, Griscelli syndrome, Hailey-Hailey disease, Harlequin type ichthyosis, Hemochromatosis, hereditary, Hemophilia, Hepatoerythropoietic porphyria, Hereditary coproporphyria, Hereditary hemorrhagic telangiectasia (Osler-Weber-Rendu syndrome), Hereditary inclusion body myopathy, Hereditary multiple exostoses, Hereditary spastic paraplegia (infantile-onset ascending hereditary spastic paralysis), Hermansky-Pudlak syndrome, Hereditary neuropathy with liability to pressure palsies, Heterotaxy, Homocystinuria, Huntington's disease, Hunter syndrome, Hurler syndrome, Hutchinson-Gilford progeria syndrome, Hyperlysinemia, Hyperoxaluria, primary, Hyperphenylalaninemia, Hypoalphalipoproteinemia (Tangier disease), Hypochondrogenesis, Hypochondroplasia, Immunodeficiency-centromeric instability-facial anomalies syndrome (ICF syndrome), Incontinentia pigmenti, lschiopatellar dysplasia, Isodicentric, Jackson-Weiss syndrome, Joubert syndrome, Juvenile primary lateral sclerosis (JPLS), Keloid disorder, Kniest dysplasia, Kosaki overgrowth syndrome, Krabbe disease, Kufor-Rakeb syndrome, LCAT deficiency, Lesch-Nyhan syndrome, Li-Fraumeni syndrome, Limb-Girdle Muscular Dystrophy, Lynch syndrome, lipoprotein lipase deficiency, Malignant hyperthermia, Maple syrup urine disease, Marfan syndrome, Maroteaux-Lamy syndrome, McCune-Albright syndrome, McLeod syndrome, MEDNIK syndrome, Mediterranean fever, Menkes disease, Methemoglobinemia, Methylmalonic acidemia, Micro syndrome, Microcephaly, Morquio syndrome, Mowat-Wilson syndrome, Muenke syndrome, Multiple endocrine neoplasia type 1 (Wermer's syndrome), Multiple endocrine neoplasia type 2, Muscular dystrophy, Muscular dystrophy (Duchenne and Becker type), Myostatin-related muscle hypertrophy, myotonic dystrophy, Natowicz syndrome, Neurofibromatosis type I, Neurofibromatosis type II, Niemann-Pick disease, Nonketotic hyperglycinemia, Nonsyndromic deafness, Noonan syndrome, Norman-Roberts syndrome, Ogden syndrome, Omenn syndrome, Osteogenesis imperfecta, Pantothenate kinase-associated neurodegeneration, Patau syndrome (Trisomy 13), PCC deficiency (propionic acidemia), Porphyria cutanea tarda (PCT), Pendred syndrome, Peutz-Jeghers syndrome, Pfeiffer syndrome, Phenylketonuria, Pipecolic acidemia, Pitt-Hopkins syndrome, Polycystic kidney disease, Polycystic ovary syndrome (PCOS), Porphyria, Prader-Willi syndrome, Primary ciliary dyskinesia (PCD), Primary pulmonary hypertension, Protein C deficiency, Protein S deficiency, Pseudo-Gaucher disease, Pseudoxanthoma elasticum, Retinitis pigmentosa, Rett syndrome, Roberts syndrome, Rubinstein-Taybi syndrome (RSTS), Sandhoff disease, Sanfilippo syndrome, Schwartz-Jampel syndrome, Sjogren-Larsson syndrome, Spondyloepiphyseal dysplasia congenita (SED), Shprintzen-Goldberg syndrome, Sickle cell anemia, Siderius X-linked mental retardation syndrome, Sideroblastic anemia, Sly syndrome, Smith-Lemli-Opitz syndrome, Smith-Magenis syndrome, Snyder-Robinson syndrome, Spinal muscular atrophy, Spinocerebellar ataxia (types 1-29), SSB syndrome (SADDAN), Stargardt disease (macular degeneration), Stickler syndrome, Strudwick syndrome (spondyloepimetaphyseal dysplasia, Strudwick type), Tay-Sachs disease, Tetrahydrobiopterin deficiency, Thanatophoric dysplasia, Treacher Collins syndrome, Tuberous sclerosis complex, Turner syndrome, Usher syndrome, Variegate porphyria, von Hippel-Lindau disease, Waardenburg syndrome, Weissenbacher-ZweymUller syndrome, Williams syndrome, Wilson disease, Woodhouse-Sakati syndrome, Wolf-Hirschhorn syndrome, Xeroderma pigmentosum, X-linked intellectual disability and macroorchidism (fragile X syndrome), X-linked spinal-bulbar muscle atrophy (spinal and bulbar muscular atrophy), Xp11.2 duplication syndrome, X-linked severe combined immunodeficiency (X-SCID), X-linked sideroblastic anemia (XLSA), 47,XXX (triple X syndrome), XXXX syndrome (48, XXXX), XXXXX syndrome (49, XXXXX), XYY syndrome (47,XYY), and Zellweger syndrome.

35. The method of claim 28, wherein said administering step is further defined as administering a first Hypo-BioNV targeting a first biomarker, and a second Hypo-BioNV targeting a second biomarker.

36. A method of treating a central nervous system (CNS) disease, including the steps of:
administering Hypo-BioNVs to an individual;
targeting CNS cells; and
treating the CNS disease.

37. The method of claim 36, wherein the Hypo-BioNVs include rabies viral glycoprotein (RVG) peptide, and further including the step of the Hypo-BioNVs targeting the central nervous system.

38. The method of claim 36, wherein the CNS disease or disorder is chosen from the group consisting of abulia, achromatopsia, agraphia, akinetopsia, alcoholism, alien hand syndrome, Allan-Herndon-Dudley syndrome, alternating hemiplegia of childhood, Alzheimer's disease, amaurosis fugax, amnesia, amyotrophic lateral sclerosis, aneurysm, Angelman syndrome, anosognosia, aphasia, aphantasia, apraxia, arachnoiditis, Arnold-Chiari malformation, Asomatognosia, Asperger syndrome, ataxia, ATR-16 syndrome, attention deficit hyperactivity disorder, auditory processing disorder, autism spectrum disorder, Behget's disease, Bell's palsy, bipolar disorder, blindsight, brachial plexus injury, brain injury, brain tumor, Brody myopathy, Canavan disease, Capgras delusion, carpal tunnel syndrome, causalgia, central pain syndrome, central pontine myelinolysis, centronuclear myopathy, cephalic disorder, cerebral aneurysm, cerebral arteriosclerosis, cerebral atrophy, cerebral autosomal dominant arteriopathy with subcortical infarcts and leukoencephalopathy, cerebral dysgenesis-neuropathy-ichthyosis-keratoderma syndrome, cerebral gigantism, cerebral palsy, cerebral vasculitis, cerebrospinal fluid leak, cervical spinal stenosis, Charcot-Marie-Tooth disease, Chiari malformation, chorea, chronic fatigue syndrome, chronic inflammatory demyelinating polyneuropathy, chronic pain, cluster headache, Cockayne syndrome, Coffin-Lowry syndrome, coma, complex regional pain syndrome, compression neuropathy, congenital distal spinal muscular atrophy, congenital facial diplegia, corticobasal degeneration, cranial arteritis, craniosynostosis, Creutzfeldt-Jakob disease, cumulative trauma disorders, Cushing's syndrome, cyclic vomiting syndrome, cyclothymic disorder, cytomegalic inclusion body disease, cytomegalovirus infection, Dandy-Walker syndrome, Dawson disease, De Morsier's syndrome, Dejerine-Klumpke palsy, Dejerine-Sottas disease, delayed sleep phase disorder or syndrome, dementia, depression, dermatomyositis, developmental coordination disorder, diabetic neuropathy, diffuse sclerosis, diplopia, disorders of consciousness, distal hereditary motor neuropathy type V, distal spinal muscular atrophy type 1, distal spinal muscular atrophy type 2, Down syndrome, Dravet syndrome, Duchenne muscular dystrophy, dysarthria, dysautonomia, dyscalculia, dysgraphia, dyskinesia, dyslexia, dystonia, empty sella syndrome, encephalitis, encephalocele, encephalopathy, encephalotrigeminal angiomatosis, encopresis, enuresis, epilepsy, epilepsy-intellectual disability in females, Erb's palsy, erythromelalgia, essential tremor, exploding head syndrome, Fabry's disease, Fahr's syndrome, fainting, familial spastic paralysis, fetal alcohol syndrome, febrile seizures, Fisher syndrome, fibromyalgia, Foville's syndrome, fragile X syndrome, fragile X-associated tremor/ataxia syndrome, Friedreich's ataxia, frontotemporal dementia, functional neurological symptom disorder, Gaucher's disease, generalized anxiety disorder, generalized epilepsy with febrile seizures plus, Gerstmann's syndrome, giant cell arteritis, giant cell inclusion disease, globoid cell leukodystrophy, gray matter heterotopia, Guillain-Barré syndrome, head injury, headache, Hemicrania Continua, hemifacial spasm, hemispatial neglect, hereditary motor neuropathies, hereditary spastic paraplegia, heredopathia atactica polyneuritiformis, herpes zoster, herpes zoster oticus, Hirayama syndrome, Hirschsprung's disease, Holmes-Adie syndrome, holoprosencephaly, HTLV-1 associated myelopathy, Huntington's disease, hydranencephaly, hydrocephalus, hypercortisolism, hypoalgesia, hypoesthesia, hypoxia, immune-mediated encephalomyelitis, inclusion body myositis, incontinentia pigmenti, Refsum disease, infantile spasms, inflammatory myopathy, intracranial cyst, intracranial hypertension, isodicentric 15, Joubert syndrome, Karak syndrome, Kearns-Sayre syndrome, Kinsbourne syndrome, Kleine-Levin syndrome, Klippel Feil syndrome, Krabbe disease, Kufor-Rakeb syndrome, Kugelberg-Welander disease, Lafora disease, Lambert-Eaton myasthenic syndrome, Landau-Kleffner syndrome, lateral medullary (Wallenberg) syndrome, learning disabilities, Leigh's disease, Lennox-Gastaut syndrome, Lesch-Nyhan syndrome, leukodystrophy, Leukoencephalopathy with vanishing white matter, Lewy body dementia, lissencephaly, locked-in syndrome, lumbar disc disease, lumbar spinal stenosis, lupus erythematosus-neurological sequelae, Lyme disease, Machado-Joseph disease, macrencephaly, macropsia, Mal de debarquement, megalencephalic leukoencephalopathy with subcortical cysts, megalencephaly, Melkersson-Rosenthal syndrome, Menieres disease, meningitis, Menkes disease, metachromatic leukodystrophy, microcephaly, micropsia, migraine, Miller Fisher syndrome, Mini-stroke (transient ischemic attack), misophonia, mitochondrial myopathy, Mobius syndrome, monomelic amyotrophy, Morvan syndrome, motor skills disorder, Moyamoya disease, mucopolysaccharidoses, multifocal motor neuropathy, multi-infarct dementia, multiple sclerosis, multiple system atrophy, muscular dystrophy, myalgic encephalomyelitis, myasthenia gravis, myelinoclastic diffuse sclerosis, myoclonic encephalopathy of infants, myoclonus, myopathy, myotonia congenita, myotubular myopathy, narcolepsy, Neuro-Behget's disease, neurofibromatosis, neuroleptic malignant syndrome, neuromyotonia, neuronal ceroid lipofuscinosis, neuronal migration disorders, neuropathy, neurosis, Niemann-Pick disease, non-24-hour sleep-wake disorder, nonverbal learning disorder, occipital neuralgia, occult spinal dysraphism sequence, Ohtahara syndrome, olivopontocerebellar atrophy, opsoclonus myoclonus syndrome, optic neuritis, orthostatic hypotension, O'Sullivan-McLeod syndrome, otosclerosis, overuse syndrome, palinopsia, PANDAS, pantothenate kinase-associated neurodegeneration, paramyotonia congenita, paresthesia, Parkinson's disease, paraneoplastic diseases, paroxysmal attacks, Parry-Romberg syndrome, Pelizaeus-Merzbacher disease, periodic paralyses, peripheral neuropathy, pervasive developmental disorders, phantom limb/phantom pain, photic sneeze reflex, phytanic acid storage disease, Pick's disease, pinched nerve, pituitary tumors, polyneuropathy, PMG, polio, polymicrogyria, polymyositis, porencephaly, post-polio syndrome, postherpetic neuralgia, posttraumatic stress disorder, postural hypotension, postural orthostatic tachycardia syndrome, Prader-Willi syndrome, primary lateral sclerosis, prion diseases, progressive hemifacial atrophy, progressive multifocal leukoencephalopathy, progressive supranuclear palsy, prosopagnosia, pseudotumor cerebri, quadrantanopia, quadriplegia, rabies, radiculopathy, Ramsay Hunt syndrome type I, Ramsay Hunt syndrome type II, Rasmussen encephalitis, reflex neurovascular dystrophy, Refsum disease, REM sleep behavior disorder, repetitive stress injury, restless legs syndrome, retrovirus-associated myelopathy, Rett syndrome, Reye's syndrome, rhythmic movement disorder, Romberg syndrome, Saint Vitus dance, Sandhoff disease, Sanfilippo syndrome, Schilder's disease, schizencephaly, sensory processing disorder, septo-optic dysplasia, shaken baby syndrome, shingles, Shy-Drager syndrome, Sjogren's syndrome, sleep apnea, sleeping sickness, snatiation, Sotos syndrome, spasticity, spina bifida, spinal and bulbar muscular atrophy, spinal cord injury, spinal cord tumors, spinal muscular atrophy, spinocerebellar ataxia, split-brain, stiff-person syndrome, stroke, Sturge-Weber syndrome, stuttering, subacute sclerosing panencephalitis, subcortical arteriosclerotic encephalopathy, superficial siderosis, Sydenham's chorea, syncope, synesthesia, syringomyelia, Tardive dyskinesia, Tarlov cyst, tarsal tunnel syndrome, Tay-Sachs disease, temporal arteritis, temporal lobe epilepsy, tetanus, tethered spinal cord syndrome, thalamocortical dysrhythmia, Thomsen disease, thoracic outlet syndrome, Tic Douloureux, tinnitus, Todd's paralysis, Tourette syndrome, toxic encephalopathy, transient ischemic attack, transmissible spongiform encephalopathies, transverse myelitis, traumatic brain injury, tremor, trichotillomania, trigeminal neuralgia, tropical spastic paraparesis, trypanosomiasis, tuberous sclerosis, Unverricht-Lundborg disease, vestibular schwannoma, Viliuisk encephalomyelitis, visual snow, Von Hippel-Lindau disease, Wallenberg's syndrome, Wernicke's encephalopathy, West syndrome, whiplash, Williams syndrome, Wilson's disease, Y-Linked hearing impairment, and Zellweger syndrome.

39. The method of claim 36, wherein the hypo-BioNVs include a psychedelic chosen from the group consisting of lysergic acid diethylamide (LSD), psilocybin, psilocin, mescaline, 5-methoxy-N,N-dimethyltryptamine (5-MeO-DMT), dimethyltryptamine (DMT), 2,5-dimethoxy-4-iodoamphetamine (DOI), 2,5-dimethoxy-4-bromoamphetamie (DOB), ibogaine, ketamine, salts thereof, tartrates thereof, solvates thereof, isomers thereof, analogs thereof, homologues thereof, and deuterated forms thereof.
